(19) United States
(12) Patent Application Publication
(10) Pub. No.: US 2013/0196441 A1
(43) Pub. Date: Aug. 1, 2013

(54) ELECTROPORATION ELECTRODE CONFIGURATION AND METHODS

(71) Applicants: Boris Rubinsky, El Cerrito, CA (US); Gregory D. Troszak, Berkeley, CA (US)

(72) Inventors: Boris Rubinsky, El Cerrito, CA (US); Gregory D. Troszak, Berkeley, CA (US)

(73) Assignee: THE REGENTS OF THE UNIVERSITY OF CALIFORNIA, Oakland, CA (US)

(21) Appl. No.: 13/700,384

(22) PCT Filed: May 31, 2011
(86) PCT No.: PCT/US11/38606

§ 371 Date: Mar. 4, 2013

Related U.S. Application Data
(60) Provisional application No. 61/351,235, filed on Jun. 3, 2010.
(60) Provisional application No. 61/470,975, filed on Apr. 1, 2011.

(51) Int. Cl. C12N 13/00

(52) U.S. Cl. CPC C12N 13/00
USPC 435/471; 435/173.6; 435/285.2; 204/280; 204/229.5; 205/701

(57) ABSTRACT

Provided herein are the concept that “singularity-based configuration” electrodes design and method can produce in an ionic substance local high electric fields with low potential differences between electrodes. The singularity-based configuration described here includes: an anode electrode; a cathode electrode; and an insulator disposed between the anode electrode and the cathode electrode. The singularity-based electrode design concept refers to electrodes in which the anode and cathode are adjacent to each other, placed essentially co-planar and are separated by an insulator. The essentially co-planar anode/insulator/cathode configuration bound one surface of the volume of interest and produce desired electric fields locally, i.e., in the vicinity of the interface between the anode and cathode. In an ideal configuration, the interface dimension between the anode and the cathode tends to zero and becomes a point of singularity.

### CROSS REFERENCE TO RELATED APPLICATIONS

This application is a 371 National Phase of International Patent Application Serial No. PCT/US2011/038606 filed May 31, 2011 which application claims the benefit of priority under 35 U.S.C. §119(e) to U.S. Provisional Application Nos. 61/351,235, filed Jun. 3, 2010 and 61/470,975, filed Apr. 1, 2011; which are incorporated herein by reference in their entirety noting that the current application controls to the extent there is any contradiction with any earlier application and to which applications we claim priority under 35 USC §120.

### BACKGROUND OF THE INVENTION

Electroporation is the permeabilization of the cell membrane lipid bilayer due to an electric field. Although the physical mechanism that causes electroporation is not fully understood, it is believed that electroporation inducing electric fields significantly increase the potential difference at the cell membrane, resulting in the formation of transient or permanent pores. The extent of pore formation primarily depends on the strength and duration of the pulsed electric field, causing membrane permeabilization to be reversible or irreversible, as a function of the strength and temporal parameters of the electroporation inducing electric fields. Reversible electroporation is commonly used to transfer macro-molecules such as proteins, DNA, and drugs into cells, while the destructive nature of irreversible electroporation makes it suitable for pasteurization or sterilization.

Typical electric fields strength required for reversible electroporation range from about 100 V/cm to 450 V/cm. In irreversible electroporation the required electric fields can range from 200 V/cm to as high as 60,000 V/cm.

Typical electroporation devices have electrodes (E) that roughly face one another, as shown in FIG. 1. In typical electroporation procedures, the targeted cells are placed between the electrodes and pulsed voltages or currents, or alternating voltages or currents, are applied on the electrodes in order to induce the required electroporation electric field in the volume between the electrodes. The relevant electroporation electric field that is produced is roughly proportional to the potential difference between the electroporation electrodes and inversely proportional to the distance (d) between electrodes (E). In such typical electroporation electrode configurations, the distance between the electrodes is constrained by the order of magnitude of the size of the cells to be electroporated or by the size of the volume to be electroporated. When high fields are required, such as in irreversible electroporation, the conventional design principles lead to the need for high potential differences across the electroporation electrodes. Large potential differences between electrodes have drawbacks. These include the need for power supplies that are able to produce these large potential differences and deliver them in a precise mode. These devices can be expensive to fabricate and energy wasteful. Furthermore, the potential differences required for large electric fields are often large enough to cause water electrolysis, resulting in electrode depletion and bubble formation, or electric discharges all of which adversely affect the electroporation process.

It would be desirable to develop an electrode configuration that can deliver high electric fields with low potential differences between electrodes.

### BRIEF SUMMARY OF THE INVENTION

Presented herein is a new electrode design principle that can achieve high electric fields with low potential differences between the electrodes. The central idea is that high fields are produced at points of singularity. Therefore, electrode configurations that produce points of singularity can generate high fields with low potential differences between the electrodes.

Provided herein are the concept that “singularity-based configuration” electrodes design and method can produce in an ionic substance local high electric fields with low potential differences between electrodes. The singularity-based configuration described here includes: an anode electrode; a cathode electrode; and an insulator disposed between the anode electrode and the cathode electrode. The singularity-based electrode design concept refers to electrodes in which the anode and cathode are adjacent to each other, placed essentially co-planar and are separated by an insulator. The essentially co-planar anode/insulator/cathode configuration bound one surface of the volume of interest and produce desired electric fields locally, i.e., in the vicinity of the interface between the anode and cathode. In an ideal configuration, the interface dimension between the anode and the cathode tends to zero and becomes a point of singularity.

An example of one possible method to use the singularity-based electrode configuration include a device for electroporation: (1) providing a channel including a series of co-planar anode electrodes and cathode electrodes, wherein adjacent anode electrodes and cathode electrodes are separated by an insulator; (2) flowing an electrolyte through the micro-electroporation channel; (3) flowing a cell through the micro-electroporation channel; and (4) applying a potential difference between adjacent anode electrodes and cathode electrodes. Other electroporation configurations using the singularity-based electrode configuration are possible. Other applications to localized high fields with singularity-based electrodes are also possible

### BRIEF DESCRIPTION OF THE FIGURES

The accompanying drawings, which are incorporated herein, form part of the specification. Together with this written description, the drawings further serve to explain the principles of, and to enable a person skilled in the relevant art(s), to make and use the systems and methods presented. In the drawings, like reference numbers indicate identical or functionally similar elements.

FIG. 1 is a schematic diagram of a typical electroporation electrode configuration.

FIG. 2A is a schematic illustration of electric field streamlines in a micro-electroporation configuration, having adjacent electrodes separated by a small insulator.

FIG. 2B is a schematic illustration of an electrode configuration, in accordance with one embodiment presented herein.

FIG. 3 is a schematic illustration of the preparation of an electrode configuration, in accordance with one embodiment presented herein.

FIG. 4(a) is a schematic of the micro-electroporation channel configuration.

FIG. 4(b) illustrates a model domain in the absence of a cell.

FIG. 4(c) illustrates a model domain in the presence of a cell.

FIG. 5 shows radially-varying electric fields generated in the micro-electroporation channel.

FIG. 6 shows how larger electric field magnitudes are present in micro-electroporation channels with smaller heights.

FIG. 7 shows large dimensionless electric field contours are more focused and span the entire height of the micro-electroporation channel for small values of A.

FIG. 8 shows how, in the presence of a cell, dimensionless electric field contours are compacted due to the insulating cell membrane.

FIG. 9 illustrates how cells experience exponentially greater dimensionless electric field magnitudes as cell radius increases.

FIG. 10 shows a temperature distribution in model domain.

FIG. 11 shows flowing electrolyte velocity arrows in model domain.

FIG. 12 shows Enterotoxigenic Escherichia coli (ETEC, a type of E. coli) cells flowing through a 0.6 μm high micro-electroporation channel with a 0.1 V potential between the electrodes.

FIG. 13 shows yeast cells flowing through a 4.2 μm high micro-electroporation channel with a 0.1 V potential between the electrodes.

FIG. 14 shows the electric field as a function of height (Y) from the surface at the centerline of the insulating length for decreasing dimensionless insulator lengths.

FIG. 15 shows the electric field developed across an E. Coli bacteria as it flows past an insulator of 100 nanometers in a channel.

FIG. 16 shows the electric field developed across a yeast cell as it flows past an insulator of 100 nanometers in a channel.

FIG. 17 is a table showing secondary current distribution model paramenters.

FIG. 18 shows non-dimensional electric field (NDE) magnitudes at X=0.5, Y=1 for various relative insulator thincknesses (I) and domain aspect ratios (A).

FIG. 19 shows electric field magnitudes along a centerline directly above the insulator in the secondary current distribution model.

FIG. 20 shows how power input to the singularity-induced micro-electroporation configuration depends on applied voltage and water conductivity.

FIG. 21 shows a galvanic electroporation device

FIG. 22 shows a schematic of the secondary current distribution model domain.

FIG. 23 shows an electric field magnitude along the y-centerline.

FIG. 24 shows power density as a function of load voltage.

### DETAILED DESCRIPTION OF THE INVENTION

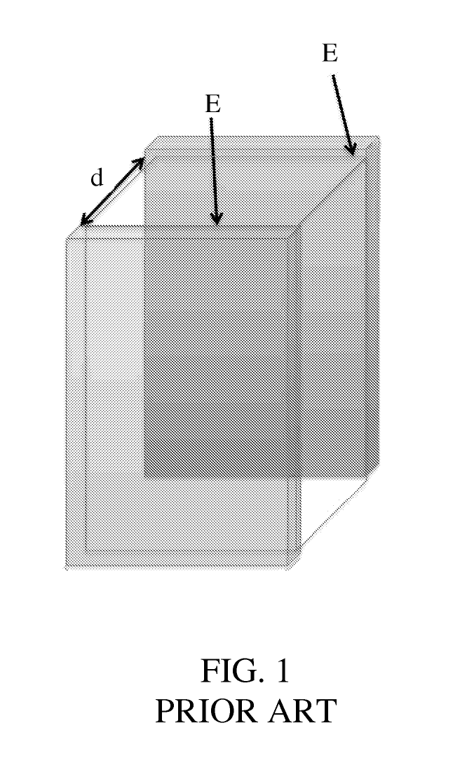

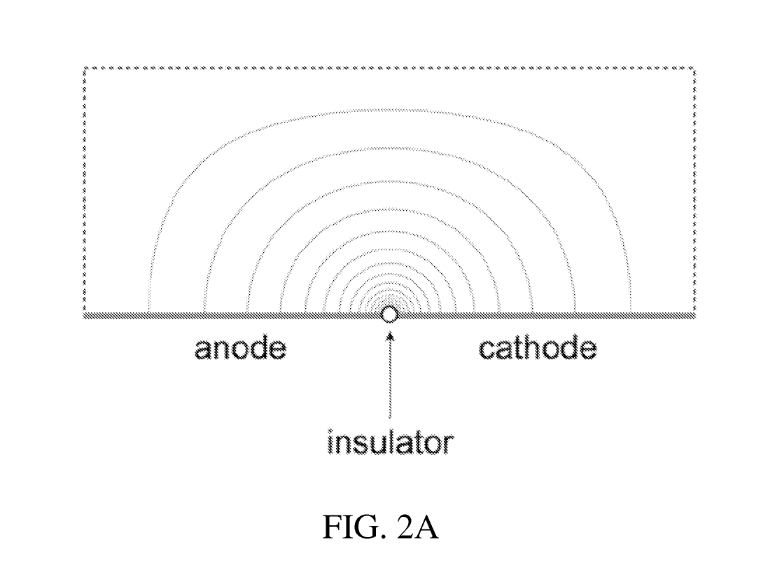

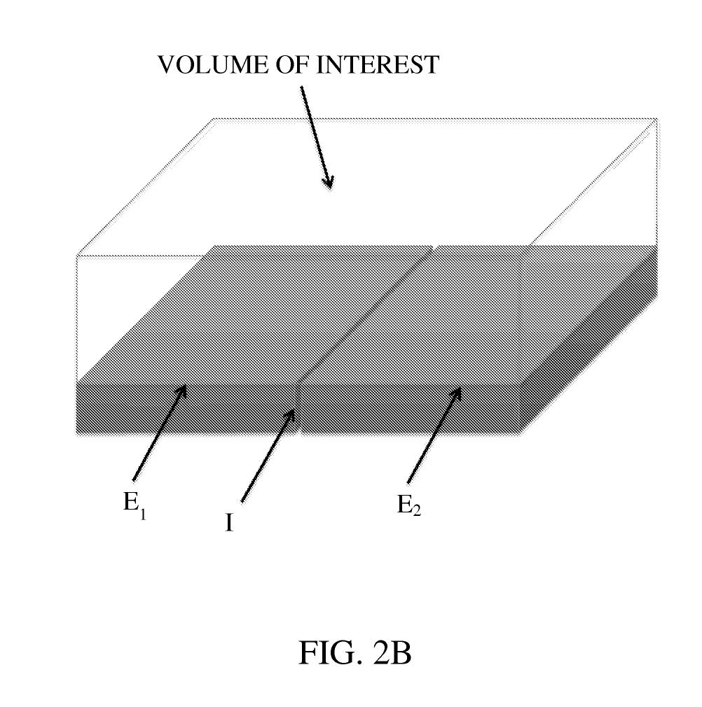

Presented herein is a singularity-based electrode configuration, which enables the generation of a local high-strength electric field in an electrolyte. The point of singularity in the context of this invention is a point in which there is a discontinuity in the potential distribution in or around and in contact with the domain of interest. At the design limit, this discontinuity has a geometrical dimension of zero. Comparison between FIG. 1 and FIGS. 2A and 2B illustrates the difference between previous electrode design concepts (FIG. 1) and the present concept (FIGS. 2A and 2B), respectively. FIG. 1 shows a typical configuration designed to produce an electric field in a volume of an electrolyte. In the typical configuration the volume of interest is confined between the electrodes. The electric field is directly proportional to the voltage difference between the electrodes and inversely proportional to the distance between the electrodes. It is possible to increase the electric field in the volume of interest by reducing the distance between the electrodes and/or by increasing the potential difference between the electrodes. In principle an infinite electric field can be produced by a finite potential difference between the electrodes, at the limit, when the distance between the electrodes goes to zero. However, since the volume of interest is between the electrodes, there is no utility for a configuration in which the distance between the electrodes is zero.

The new design concept shown in FIGS. 2A and 2B suggests that the two electrodes be placed essentially on the same plane, bounding a surface of the electrolyte volume of interest. The anode and the cathode are separated by an insulating gap. In this configuration the local electric field at the interface between the electrolyte and anode/insulator/cathode is also a function of the dimension of the insulator and the potential difference between the anode and cathode. However, in this configuration, the volume of interest is bounded on the outer surface by the electrodes and not confined between the electrodes. Therefore in an ideal configuration as the limit of the insulator dimension goes to zero, the interface between the electrodes becomes a point of singularity and in the electrolyte, infinitesimally final differences of potential between the electrodes can produce an infinitely high field at the point of singularity. This configuration facilitates therefore the generation of very high electric fields in a volume of interest using small potential differences. FIG. 2A demonstrates the utility of this design by showing lines of constant electric fields emanating from a point of singularity between two electrodes. FIG. 2A shows that the volume affected by the singularity-based electrodes is substantial and predictable, and therefore this electrode design can be used to produce high electric fields, with low potential differences in a volume of interest.

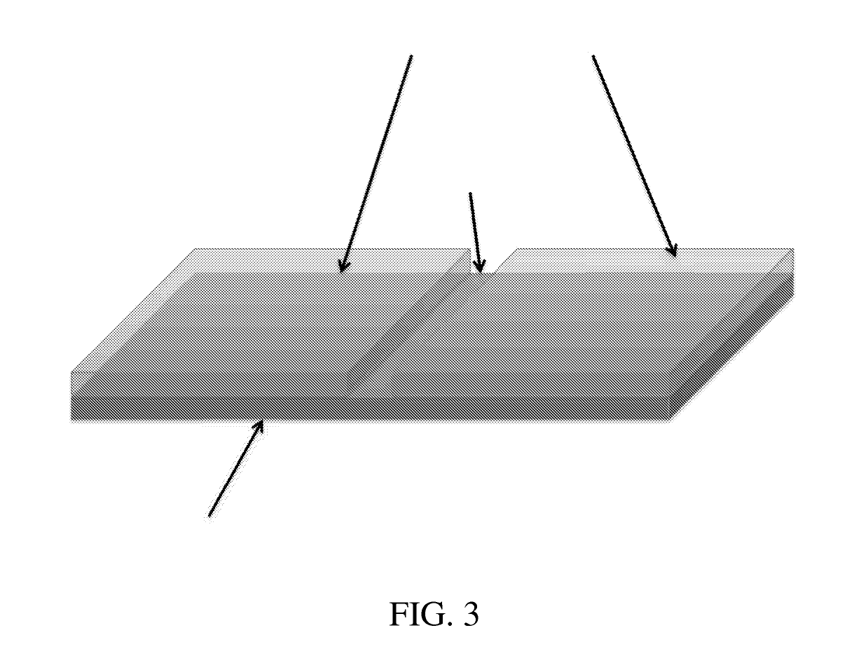

Advances in micro and nanotechnologies can be used to produce the singularity-based configuration. FIG. 3 illustrates such a design. The design is based on an electrically insulating surface, such as glass. A conductor, such as gold or platinum, is deposited by vapor deposition on the glass surface. The thickness of the deposited layer can range from single nanometers to micrometers. Generating a cut in the deposited metal, to the glass surface, produces the insulating gap between the electrodes. The electrolyte can be placed on the surface of the structure facing the two electrodes and the gap, and the high electric fields are produced in the gap.

Focused laser beams can be used to produce cuts, with widths of single microns. Numerous lithographic techniques are capable of producing sub-100 nm features, and could be used to create the insulators in a micro-electroporation channel. Immersion lithography is a photolithography enhancement technique that places a liquid with a refractive index greater than one between the final lens and wafer. Current immersion lithography tools are capable of creating feature sizes below 45 nm. Additionally, electron beam lithography, a form of lithography that uses a traveling beam of electrons, can create features smaller than 10 nm.

The design described in FIGS. 2A, 2B and 3 can be used in a variety of configurations. A typical configuration is generally composed of an electrolyte placed or flowing over two adjacent electrodes separated by a small insulator. As shown in FIG. 2A, application of a small potential difference between the adjacent electrodes results in a radially varying electric field emanating from the insulator. The electric field can be used to electroporate cells suspended in the electrolyte.

There are numerous possible designs that employ the singularity-based electrode design. For instance, it would be possible to coat a stirrer blade with such a material to retain the sterility of the blade. Or it would be possible to coat the walls of a container with such a design to maintain the sterility of the walls by producing electric fields.

While the singularity-based design is for electroporation, the advantage of having the ability to produce locally in electrolytic solutions, high electric fields with low potential differences could be used in deep brain implants, pacemakers and other medical applications.

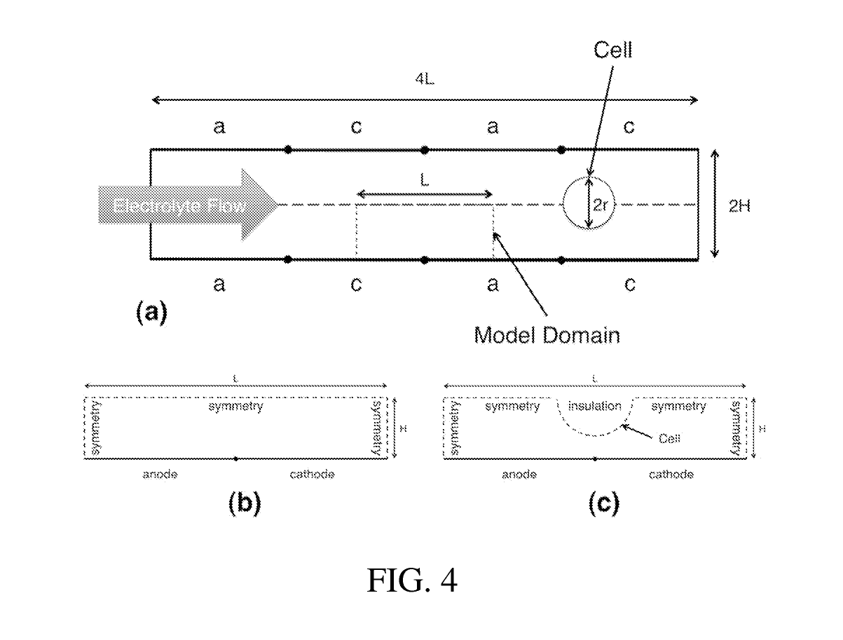

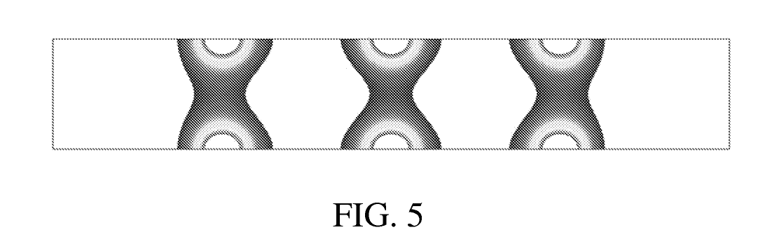

As a more detailed illustration of the various possible applications of the singularity-based electrodes, we will describe in greater detail and as an example a configuration in the form of a “micro-electroporation” channel. As shown in FIGS. 4(a) and 5, mirroring the configuration and placing it in series forms a micro-electroporation channel with multiple electric fields. Cells flowing through this channel will experience a pulsed electric field. The magnitude of this electric field can be adjusted by altering the height of the channel. Furthermore, adjusting the electrolyte flow rate alters the duration of the electric field experienced by cells suspended in the electrolyte.

A two-dimensional, steady-state, primary current distribution model was developed to understand the effect of micro-electroporation channel geometry and cell size on the electric field in the flowing electrolyte. In the absence of cells, decreasing the micro-electroporation channel height results in an exponential increase in the electric field magnitude in the center of the channel. Additionally, cells experience exponentially greater electric field magnitudes the closer they are to the micro-electroporation channel walls.

The presented micro-electroporation channel differs from traditional macro and micro-electroporation devices in several ways. In electroporation devices with facing electrodes, a cell's proximity has no bearing on the electric field magnitude it will experience. Conversely, in the micro-electroporation channel presented, the electric field magnitude experienced by a cell is dictated by the gap between the cell and the channel wall. Because of this, cell size does not affect the potential difference required to achieve a desired electric field.

Another difference between the presented micro-electroporation channel and traditional macro and micro-electroporation devices is that less electrical equipment is required. Traditional macro and micro-electroporation devices require a pulse generator and power supply. However, in the micro-electroporation channel presented, the need for a pulse generator is eliminated since it contains a series of adjacent electrodes. Furthermore, since the micro-electroporation channel presented only requires a small potential difference, a minimal power source (such as a battery) is needed.

The simplicity of electroporation makes it a powerful technology. The presented micro-electroporation channel increases the accessibility of electroporation, making its use feasible for a wide range of non-traditional applications.

In one embodiment, there is provided a micro-electroporation channel configuration. The channel configuration generally includes an anode electrode; a cathode electrode; and an insulator disposed between the anode electrode and the cathode electrode. The anode electrode, insulator, and cathode electrode are positioned co-planar along one side of the micro-electroporation channel. The configuration may further include an electrolyte flowing through the channel over the anode electrode, insulator, and cathode electrode. A flow rate control system may be provided to alternate the flow of electrolyte through the channel. In one embodiment, the insulator separates the anode electrode from the cathode electrode by less than 200 nm, or by less than 100 nm. In another embodiment, the insulator separates the anode electrode from the cathode electrode by about 100 nm. A battery power source may also be provided, avoiding the use of a pulse generator.

In another embodiment, the micro-electroporation channel configuration includes a second anode electrode positioned on the opposite side of the channel relative to the first anode electrode; a second cathode electrode positioned on the opposite side of the channel relative to the first cathode electrode; and a second insulator disposed between the second anode electrode and the second cathode electrode. The second anode electrode and the second cathode electrode are generally co-planar with respect to one another. As such, the electrode configuration creates a channel, in which a cell is passed for electroporation. In yet another embodiment, there is provided a configuration in which an ionic substance is bounded on one side by a configuration containing the singularity-based electrode configuration, in the form of a flat plate or essentially a flat plate on which an ionic substance is placed.

In another embodiment, there is provided a configuration in which the ionic substance is surrounded by the singularity-based electrode configuration in the form of a channel or container in which the ionic substance is set or through which it flows. The electric fields at the point of singularity can be suitable to produce reversible or irreversible electroporation electroporation in the cells in the ionic substances. Reversible electric fields from 50 V/cm to 1000 V/cm, 100V/cm to 450 V/cm, DC or AC. Irreversible electric fields from 50 V/cm to 100,000 V/cm, from 200 V/cm to 30 kV/cm

In still another embodiment, there is provided a method of micro-electroporation. The method generally includes: (1) providing a micro-electroporation channel including a series of co-planar anode electrodes and cathode electrodes, wherein adjacent anode electrodes and cathode electrodes are separated by an insulator; (2) flowing an electrolyte through the micro-electroporation channel; (3) flowing a cell through the micro-electroporation channel; and (4) applying a potential difference between adjacent anode electrodes and cathode electrodes. The method may further include: (5) alternating the flow rate of the electrolyte through the micro-electroporation channel; and (6) coupling the anode electrodes and the cathode electrodes to a battery power source. Each insulator may separate the anode electrode from the adjacent cathode electrode by less than 200 nm, or by less than 100 nm, or by about 100 nm. Such method may be used for applications such as water sterilization or cell transfection.

In another embodiment, there is provided a micro-electroporation channel configuration, comprising: an anode electrode; a cathode electrode; and an insulator disposed between the anode electrode and the cathode electrode, wherein the anode electrode, insulator, and cathode electrode are positioned co-planar along one side of the micro-electroporation channel. An electrolyte may then be provided flowing through the channel over the anode electrode, insulator, and cathode electrode. The insulator may separate the anode electrode from the cathode electrode by between 5 nanometers and two microns. The micro-electroporation channel configuration may further comprising a power source selected from a group consisting of: a pulsed potential, an AC potential, and an electrolytic reaction involving the electrodes and an ionic solution. The ionic solution may be a physiological solution that contains cells, live tissue, or dead tissue. In one embodiment, the power source is couple to the electrodes and configured to deliver an appropriate supply of current in order to create an adjustable field. The field may be adjusted to meet the application (e.g., reversible electroporation or irreversible electroporation). In one embodiment, a field is applied for irreversible electroporation, without causing thermal damage to the cells of interest.

Traditional macro and micro-electroporation have deficiencies that are addressed by the presented micro-electroporation channel. Due to the large quantities of cells treated in macro-electroporation, the extent of cell permeabilization varies throughout the population. While micro-electroporation addresses this issue, it typically results in lower throughput. The focused electric fields in the presented micro-electroporation channel, which can be modified with channel geometry, offer better control over cell permeabilization than macro-electroporation devices. Additionally, the flow-through nature of the channel makes it suitable for treating large quantities of cells.

Another deficiency addressed by the presented micro-electroporation channel is the need for large, electrolysis-inducing potential differences in traditional macro and micro-electroporation devices. Most macro and micro-electroporation devices have facing electrodes, which results in a uniform electric field that is inversely proportional to their separation distance. Although the separation distances in micro-electroporation devices are significantly smaller than those in typical electroporation devices, they are limited by cell size. Because of this, large, electrolysis-inducing potential differences are required to generate a desired electric field. The presented micro-electroporation channel contains a series of adjacent electrodes separated by small insulators. Application of a small, non-electrolysis-inducing potential difference results in a series of radially-varying electric fields that emanate from the small insulators. Because of this, only a small power source (such as a battery) is required. Reducing the electrical equipment required makes electroporation feasible for a wider range of applications.

### Potential Applications

The non-dimensional models show that cells of assorted sizes can experience various electric field magnitudes by adjusting the micro-electroporation channel height. Furthermore, the electrolyte flow rate can be used to control exposure time. These parameters enable a great deal of control over the extent of cell permeabilization without the need for complicated electrical equipment, making this concept useful for a number of potential applications including water sterilization and cell transfection.

### Water Sterilization

Contaminated water can cause numerous diseases including diarrhea, which accounts for 4% of all deaths worldwide (2.2 million). Most of these deaths occur among children under the age of five and represent approximately 15% of all child deaths under this age in developing nations. It is estimated that sanitation and hygiene intervention could reduce diarrheal infection by one-quarter to one-third; however, this requires access to sterile water, which can be scarce, particularly in rural areas of developing nations.

Enterotoxigenic Escherichia coli (ETEC, a type of E. coli) is a 2 μm long, 0.5 μm diameter, rod-shaped fecal coliform, and is the leading bacterial cause of diarrhea in developing nations. Currently, vaccination is the most effective method of preventing diarrhea caused by ETEC. However, vaccines are not available in developing nations where ETEC is endemic.

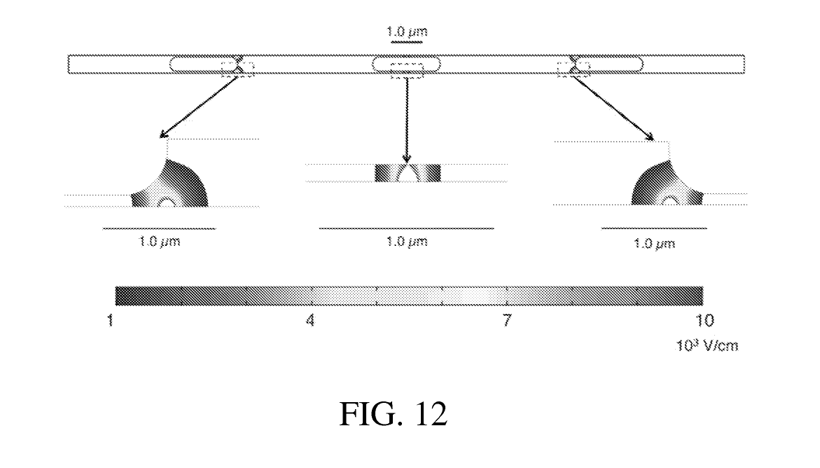

It is possible to destroy ETEC with irreversible electroporation using the concept presented herein. The results of a dimensional form of the primary current distribution model show that ETEC cells in water flowing through the center of a 0.6 μm high micro-electroporation channel with a 0.1 V potential difference between adjacent electrodes experience electric field magnitudes between 1000 and 10000 V/cm, inducing irreversible electroporation (FIG. 12). It should be noted that this is a conservative estimate, since cells flowing through the center of the channel will experience relatively low strength electric fields compared to cells flowing closer to the electrodes.

### Cell Transfection

Cell transfection is the process of introducing large molecules, primarily nucleic acids and proteins, into cells. These large molecules typically enter cells through transient pores created in the cell membrane by chemical and physical methods, such as electroporation. However, due to the bulk nature of the process, it is difficult to determine the optimal electroporation parameters for high transfection efficiency and minimal cell death. Traditional micro-electroporation could remedy this problem; however, traditional micro-electroporation is not appropriate for treating large quantities of cells.

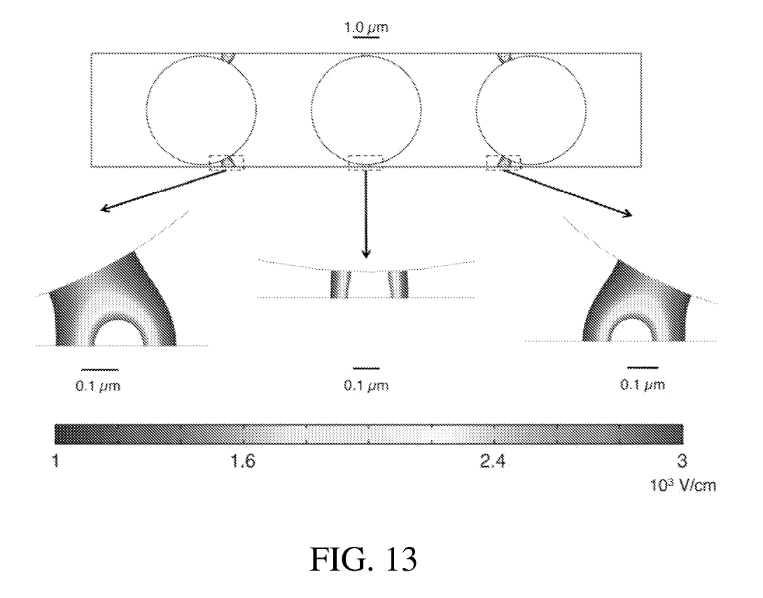

In contrast, the flow-through nature of the micro-electroporation channel presented herein makes it ideal for treating many cells while maintaining control of the electric fields they experience. Yeast is a 4 μm diameter cell widely used in genetic research because it is a simple cell that serves as a representative eukaryotic model. A dimensional form of the primary current distribution model shows that yeast cells flowing through a 4.2 μm high channel with a potential of 0.1 V between the electrodes experience reversible electroporation inducing electric field magnitudes, creating the transient pores needed for cell transfection (FIG. 13). By stacking multiple micro-electroporation channels atop one another, it would be possible to increase throughput while maintaining consistent electric fields.

### EXAMPLES

The following paragraphs serve as example embodiments of the above-described systems. The examples provided are prophetic examples, unless explicitly stated otherwise.

### Example 1

### Nomenclature for Example 1

φ=electric potential
φₐ=electric potential at anode
φ_{c}=electric potential at cathode
φ_{diff}=electric potential difference between electrodes
L=active electrode length
H=half of micro-electroporation channel height
r=cell radius
Φ=non-dimensional electric potential
Φₐ=non-dimensional electric potential at anode
Φ_{c}=non-dimensional electric potential at cathode
X=non-dimensional x-coordinate
Y=non-dimensional y-coordinate
A=channel aspect ratio
R=relative cell radius
E=non-dimensional electric field
T=temperature
Q_{gen}=volumetric heat generation
k=thermal conductivity
ρ=density
Cₚ=specific heat at constant pressure
u=x-velocity
σ=electrical conductivity
μ=dynamic viscosity
p=pressure

FIG. 4(a) is a schematic of the micro-electroporation channel configuration. FIG. 4(b) illustrates a model domain in the absence of a cell. FIG. 4(c) illustrates a model domain in the presence of a cell. FIG. 5 shows radially-varying electric fields generated in the micro-electroporation channel. A two-dimensional, steady-state, primary current distribution model was developed to understand the effect of micro-electroporation channel geometry and cell size on the electric field in the flowing electrolyte. Primary current distribution models neglect surface and concentration losses at the electrode surfaces, only taking into account electric field effects from ohmic losses in the electrolyte. Therefore, primary current distribution models are governed by the Laplace equation:

∇²φ=0

where φ is the electric potential. Furthermore, electrode surfaces are assumed to be at a constant potential, making the boundary conditions at the adjacent electrode surfaces:

φₐ=φ_{diff} for {0<x≦L/2 y=0}

φ_{c}=0 for {L/2<x≦L y=0}

where φₐ and φ_{c} are the potentials at the anode and cathode, respectively, φ_{diff} is the potential difference between them, and L is the active electrode length. The remaining symmetry boundaries are governed by:

$$∇ φ = 0 for \{ \begin{matrix}x = 0 & 0 < y \leq H \\ x = L & 0 < y \leq H \\ 0 < x \leq L & y = H\end{matrix} \}$$

where H is half of the height of the micro-electroporation channel. Due to the insulating properties of cell membranes, cells flowing through the micro-electroporation channel are modeled as electrically insulating boundaries, which are identical to symmetry boundaries.

### Non-Dimensionalization of the Primary Current Distribution Model.

The primary current distribution model was non-dimensionalized to analyze the effect of micro-electroporation channel geometry and cell size on electric fields in the electrolyte. The Laplace equation in two-dimensional Cartesian coordinates is:

$$\frac{{\partial }^{2} φ}{\partial {x}^{2}} + \frac{{\partial }^{2} φ}{\partial {y}^{2}} = 0$$

Substituting the non-dimensional variables:

Φ=φ/φ_{diff}

X=x/L

Y=y/H

into the Laplace equation yields a non-dimensional form:

$$\frac{{\partial }^{2} Φ}{\partial {X}^{2}} + {( \frac{L}{H} )}^{2} \frac{{\partial }^{2} Φ}{\partial {Y}^{2}} = 0$$

Defining the non-dimensional geometry parameter (channel aspect ratio):

$$A = \frac{H}{L}$$

the non-dimensional Laplace equation becomes:

$$\frac{{\partial }^{2} Φ}{\partial {X}^{2}} + \frac{1}{{A}^{2}} \frac{{\partial }^{2} Φ}{\partial {Y}^{2}} = 0$$

Substitution of the non-dimensional variables into the boundary conditions yields:

$${Φ}_{a} = 1 for \{ \begin{matrix}0 < X \leq 0.5 & Y = 0\end{matrix} \}$$

$${Φ}_{c} = 0 for \{ \begin{matrix}0.5 < X \leq 1 & Y = 0\end{matrix} \}$$

$$∇ Φ = 0 for \{ \begin{matrix}X = 0 & 0 < Y \leq 1 \\ X = 1 & 0 < Y \leq 1 \\ 0 < X \leq 1 & Y = 1\end{matrix} \}$$

Finally, for a spherical cell, the non-dimensional cell radius (relative cell radius) is defined as:

R=r/H

where r is the cell radius.

### Solution of the Primary Current Distribution Model

The non-dimensional primary current distribution model is characterized by the channel aspect ratio (A) and the relative cell radius (R). A parametric study was performed by varying these parameters in a series of models. In each model, the non-dimensional potential distribution was solved for using the finite element analysis software COMSOL Multiphysics 3.5a. A non-dimensional electric field, defined as:

E=∇Φ

was calculated using the non-dimensional potential distribution.

Cells were initially excluded from the models to validate the finite element solution and to better understand how micro-electroporation channel geometry affects the electric field in the electrolyte. These models are only characterized by the channel aspect ratio and have a simplified geometry. This simple geometry, along with the homogenous nature of the non-dimensional Laplace equation and three symmetry boundaries enabled an analytical solution using the separation of variables method. The analytical solution was used to verify the results of the finite element solution. Once the finite element solution was verified, cells were included in the models.

### Preliminary Coupled Thermal Model

In addition to the primary current distribution model, a preliminary, two-dimensional, steady-state coupled thermal model was developed to determine the temperature distribution in the flowing electrolyte. Three models compose the coupled model: (1) a convection and conduction model, (2) the primary current distribution model, and (3) a Navier-Stokes model.

The two-dimensional, steady-state heat equation with conduction and convection in the x-direction is:

$$\frac{{\partial }^{2} T}{\partial {x}^{2}} + \frac{{\partial }^{2} T}{\partial {y}^{2}} + \frac{{Q}_{gen}}{k} - \frac{{ρC}_{p} u}{k} \frac{\partial T}{\partial x} = 0$$

where T is temperature, k is thermal conductivity, ρ is density, Cₚ is the specific heat at constant pressure, Q_{gen} is the volumetric heat generation, and u is the fluid velocity distribution in the x-direction. The volumetric heat generation term, Q_{gen}, is the result of ohmic heating in the electrolyte, and in two-dimensions is governed by:

$${Q}_{gen} = σ {\vert \frac{\partial φ}{\partial x} + \frac{\partial φ}{\partial y} \vert}^{2}$$

where σ is the electrical conductivity of the electrolyte, and the potential distribution is determined from the primary current distribution model. Additionally, the fluid velocity distribution in the x-direction, u, is determined by applying the Navier-Stokes equations to steady flow between two horizontal, infinite parallel plates, resulting in:

$$u = \frac{1}{2 μ} ( \frac{\partial p}{\partial x} ) ( {y}^{2} - {H}^{2} )$$

where μ is the dynamic viscosity of the electrolyte, and ∂p/∂x is a constant pressure gradient.

The boundary conditions of the conduction and convection model are constant temperature at the left domain boundary:

T=293K for {x=0 0<y≦H}

thermal insulation and symmetry at the bottom and centerline of the channel, respectively:

$$\frac{\partial T}{\partial y} = 0 for \{ \begin{matrix}0 < x \leq L & y = 0 \\ 0 < x \leq L & y = H\end{matrix} \}$$

and continuity at the right domain boundary:

$$\frac{\partial T}{\partial x} = 0 for \{ \begin{matrix}x = L & 0 < y \leq H\end{matrix} \}$$

The coupled thermal model was solved in COMSOL Multiphysics 3.5a for a 2 μm high (H=1 μm) 10 μm long channel with a 0.1 V potential difference between the electrodes and water as the electrolyte. The velocity profile was entered as an expression into the convection and conduction model, which used the primary current distribution model to determine the heat generation term throughout the model domain. The parameters used in the model are shown in Table 1 below.

TABLE 1

| | | | |
|---|---|---|---|
| | | | Channel |
| Potential difference | Φ_{diff} | V | 0.1 |
| Half channel height | H | μm | 1 |
| Active electrode length | L | μm | 10 |
| Pressure gradient | ∂p/∂x | Pa/μm | 100 |
| | | | Water |
| Thermal conductivity | k | W/m-K | 0.58 |
| Density | ρ | kg/m³ | 998.20 |
| Specific heat at constant pressure | Cₚ | J/kg-K | 4181.80 |
| Electrical conductivity | σ | S/m | 5.5e−6 |
| Dynamic viscosity | μ | Pa-s | 8.90e−4 |

### Primary Current Distribution Finite Element Model Verification

The non-dimensional primary current distribution finite element model was verified with an analytical solution. Correlation coefficients between the non-dimensional potential distributions of the two solutions were computed in MATLAB (R2007a version 7.4) for values of channel aspect ratio (A) between 0.1 and 1. The correlation coefficients were 1 for all values of channel aspect ratio, indicating that the finite element and analytical solutions are identical.

Non-Dimensional Primary Current Distribution Model Results without Cells

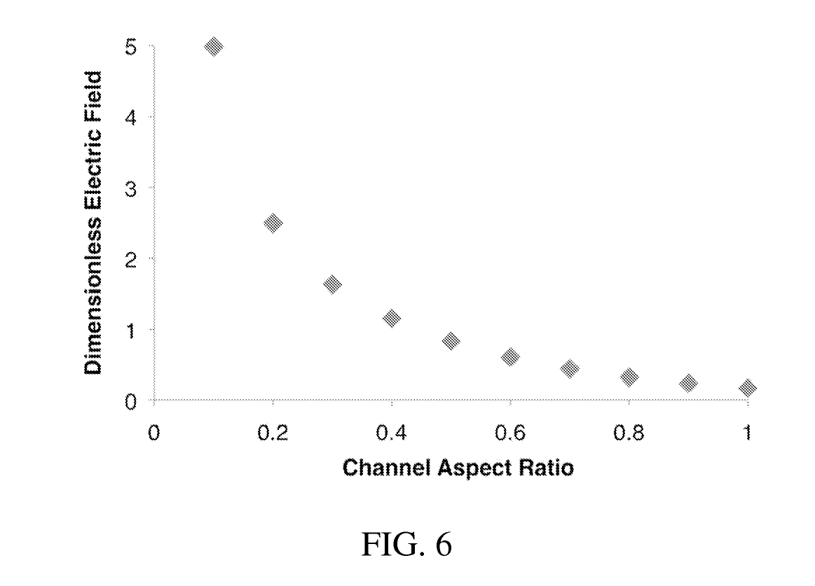

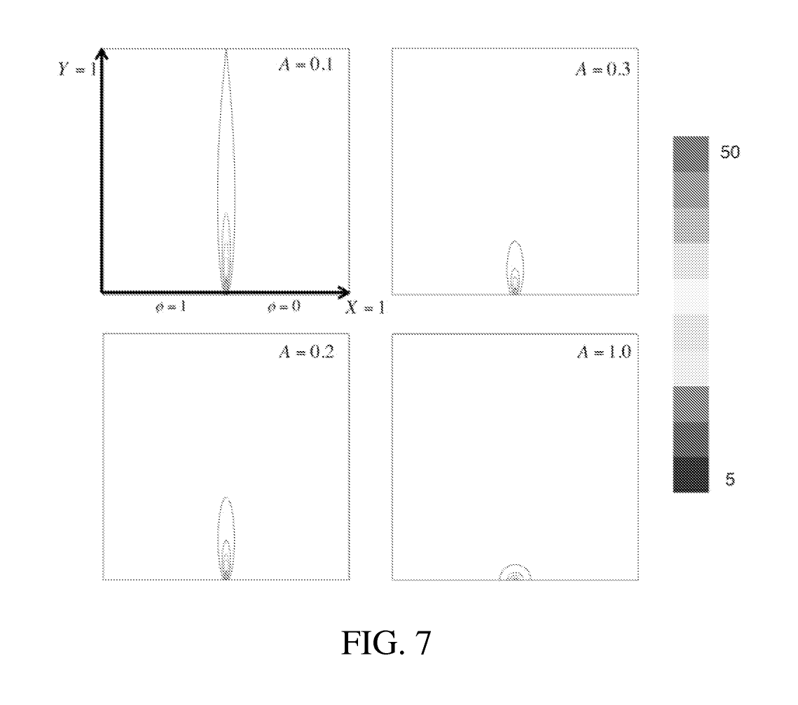

In the absence of cells, the models are only characterized by the channel aspect ratio (A). As the channel aspect ratio decreases, the magnitude of the non-dimensional electric field increases exponentially in the center of the micro-electroporation channel. FIG. 6 shows how larger electric field magnitudes are present in micro-electroporation channels with smaller heights. Furthermore, high-magnitude non-dimensional electric field contours are more focused and span the height of the channel for small channel aspect ratios. FIG. 7 shows large dimensionless electric field contours are more focused and span the entire height of the micro-electroporation channel for small values of A.

Non-Dimensional Primary Current Distribution Model Results with Cells

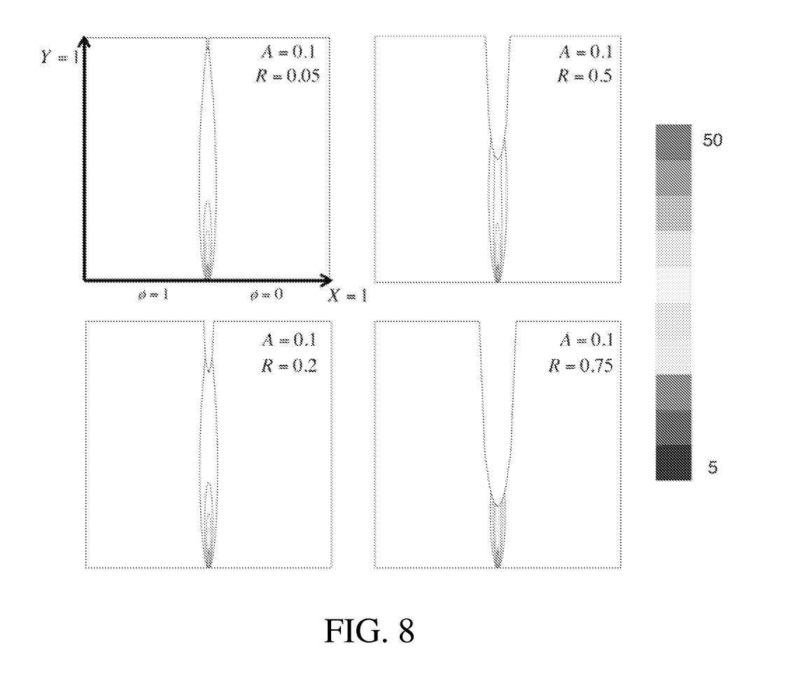

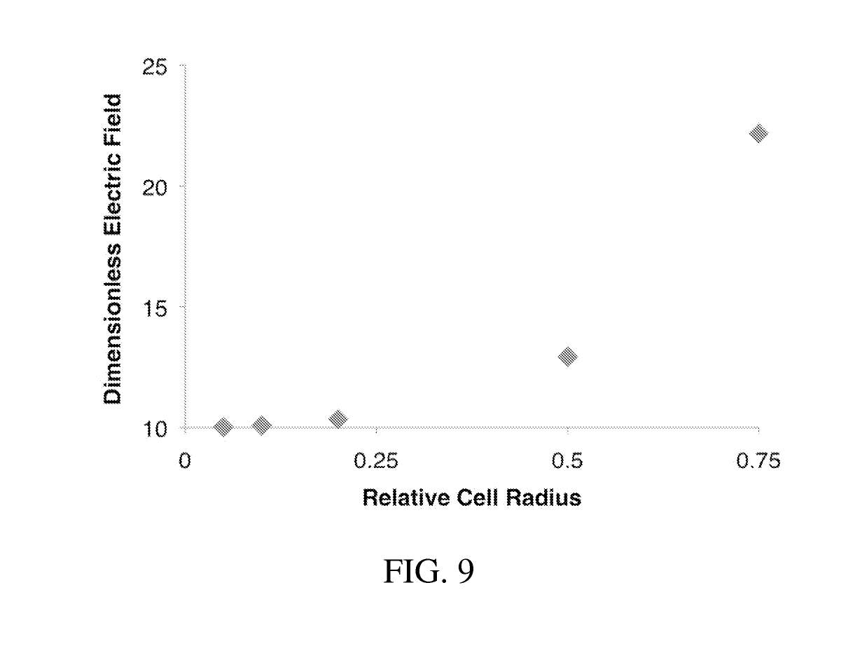

The electric field in the electrolyte is also affected by the presence of cells. Due to the insulating properties of the cell membrane, electric field contours are compacted, causing cells to experience exponentially greater electric field magnitudes as the relative cell radius increases (R). FIG. 8 shows how, in the presence of a cell, dimensionless electric field contours are compacted due to the insulating cell membrane. FIG. 9 illustrates how cells experience exponentially greater dimensionless electric field magnitudes as cell radius increases.

### Coupled Thermal Model Results

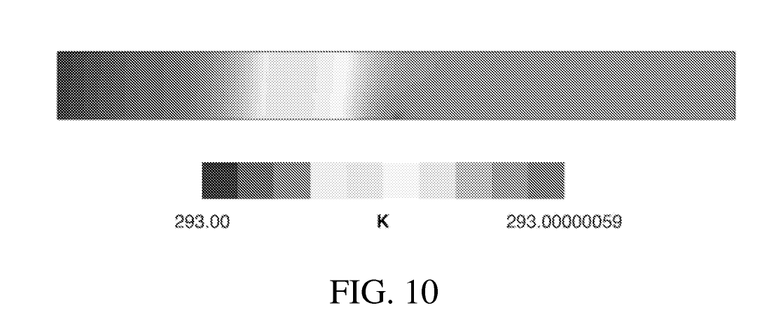

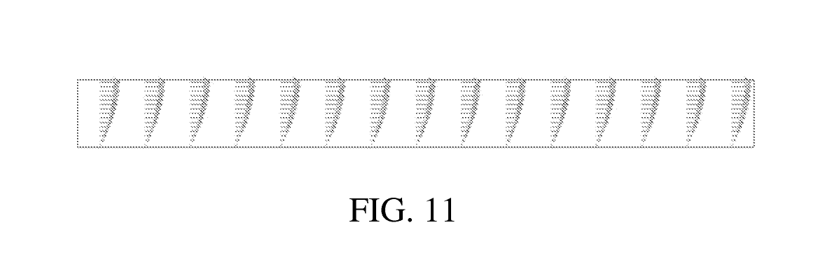

The temperature distribution in the electrolyte is shown in FIG. 10. A maximum temperature of 293.00000059 K is at the insulator and the convective heat transfer due to the electrolyte flow is apparent. Additionally, an arrow plot of the electrolyte flow is shown in FIG. 11. The maximum fluid velocity (at the center of the micro-electroporation channel) for the 1 kPa pressure difference is uₘₐₓ=0.0562 m/s.

These results show that adjusting micro-electroporation channel height is a way to control the range of electric field magnitudes in the flowing electrolyte without increasing the potential difference between the electrodes. Models with cells indicate that the closer cells are to the channel walls, the higher electric field magnitudes they will experience. Additionally, the preliminary coupled thermal model shows a 0.00000059 K temperature increase in the flowing electrolyte, which is insufficient to cause thermal cell damage.

It should be noted that changing the length of the insulator separating the adjacent electrodes would affect the electric field in the electrolyte. More specifically, electric field magnitudes throughout the electrolyte would decrease by increasing the length of the insulator.

### Example 2

The theoretical highest electric field can be produced in the configuration discussed in this invention when the dimension of the insulating singularity between the voltage sources tends to limit of zero. We have used the same methodology of analysis to evaluate what is the effect of the insulating gap thickness on the electric field produced. The results show that a technologically achievable 100 nanometer gap can produce the desired effects.

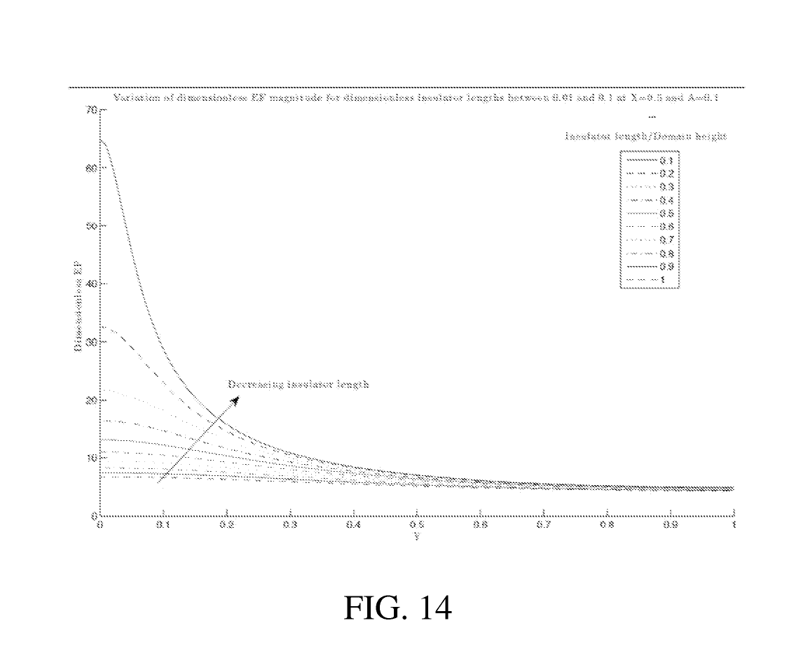

The models were done in a similar way to those described in the previous example with non-dimensional insulation lengths varying from 0.01 to 0.1 (insulation length/domain length) for an aspect ratio of 0.1. The non-dimensional insulation length can be scaled to the domain height by dividing by the aspect ratio. FIG. 14 is a plot that shows the non-dimensional electric field (EF) strength at X=0.5, for different insulation thicknesses. In other words, FIG. 14 shows the electric field as a function of height Y from the surface at the centerline of the insulating length for decreasing dimensionless insulator lengths.

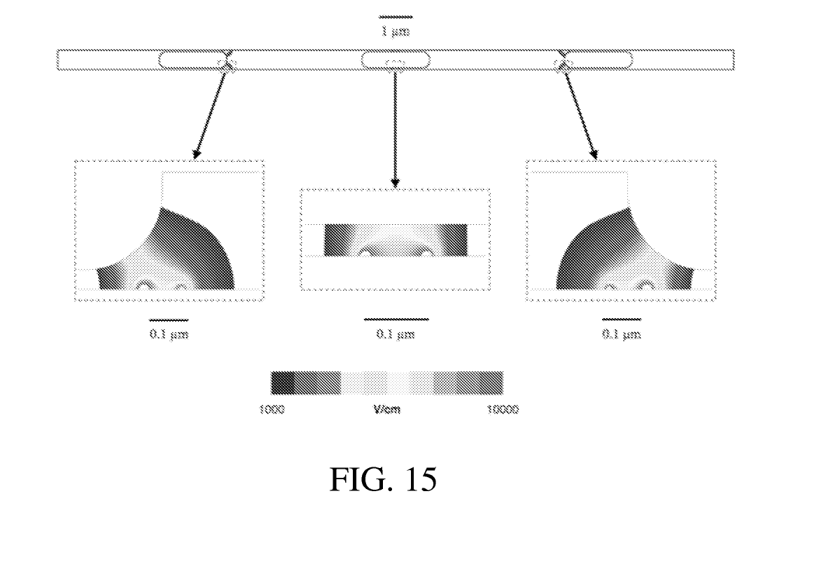

FIG. 15 shows the electric field developed across an E. Coli bacteria as it flows past an insulator of 100 nanometers in a channel. FIG. 15 shows specific applications considering a practical insulation length for the E. coli and yeast of the previous example. The results show that IRE and RE inducing electric fields are still developed with a 100 nm insulator, respectively. In this case the active electrode length is 5 μm, not that that has an effect on the results. In summary, for the E. coli models, H=0.3 μm, L=5 μm, and IL=100 nm; for the yeast models, H=2.1 μm, L=5 μm, and IL=100 nm.

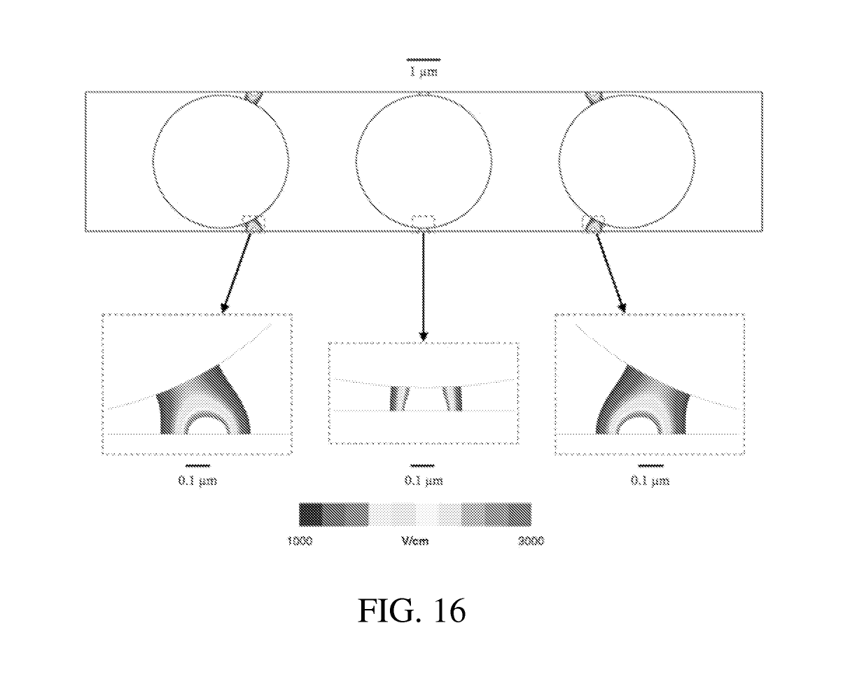

The results for yeast are given in FIG. 16. FIG. 16 shows the electric field developed across a yeast cell as it flows past an insulator of 100 nanometers in a channel.

### Example 3

This example is similar to the Example 1 and Example 2. However, Example 3 introduces a new concept. Because the voltage difference across the insulator can be very small, it can be also produced through electrolysis between two dissimilar metals separated by the insulator and brought in electric contact through the electrical conductive media. This configuration may allow for the unprecedented miniaturization of single-cell micro-electroporation devices and micro-batteries. Furthermore, while each application is independent, by combining them, it is possible to perform single-cell micro-electroporation with no power input, through electrolysis. In the process, it is even possible to produce electric power.

Electrochemical cells are devices capable of delivering electrical energy from chemical reactions (galvanic cells), or conversely, facilitating chemical reactions from the input of electrical energy (electrolytic cells). All electrochemical cells are composed of at least: (1) two electrodes where chemical reactions occur, (2) an electrolyte for ion conduction, and (3) an external conductor for continuity. Oxidation (the loss of electrons) occurs at one electrode (the anode) and reduction (the gain of electrons) occurs at the other (the cathode).

Both the anode and cathode have characteristic potentials that depend on their respective chemical reactions. The difference in these characteristic potentials dictates either the amount of work that the coupled chemical reactions can perform (galvanic cell), or the amount of work necessary to reverse the coupled chemical reactions (electrolytic cell). Thermodynamically, at constant temperature and pressure, this is described by the change in Gibb's free energy:

ΔG=−nFΔφ_{cell}

where n is the stoichiometric number of electrons transferred, F is Faraday's constant, and Δφ_{cell} is the potential difference of the coupled reactions. A negative change in Gibb's free energy implies that a chemical reaction is favorable and is able to perform work (galvanic cell). Conversely, a positive change in Gibb's free energy implies an unfavorable reaction that will need work input to proceed (electrolytic cell).

Since the Gibb's free energy is a thermodynamic quantity, it is only useful for describing systems at equilibrium. In an operating electrochemical cell a passage of current takes place, which implies that the system is not at equilibrium. The passage of current causes potential drops in the electrochemical cell, resulting in a potential difference that deviates from that observed at equilibrium. This deviation is termed overpotential and can be attributed to three types of losses: (1) surface, (2) concentration, and (3) ohmic.

Surface losses occur due to the kinetic limitations at an electrode surface. These kinetic limitations are typically governed by mass transfer, electron transfer at the electrode surface, chemical reactions preceding or following the electron transfer, and other surface reactions.

Concentration losses are caused by mass-transport limitations, which result in the depletion of charge-carriers at the electrode surface. This depletion establishes a concentration gradient between the electrode surface and bulk electrolyte, causing a potential drop.

Ohmic losses are primarily associated with ionic current flow in the electrolyte. This is governed by Ohm's law:

i=−k∇φ

where i is the ionic current, k is the electrolyte conductivity, and φ is the electric potential. Therefore, for a given current, electrolyte conductivity largely influences the ohmic potential drop in the electrolyte.

### Powerless Single Cell Micro-Electroporation

Although typical electroporation and micro-electroporation are procedurally straightforward, they both require at least a pulse generator and a power supply, which limits the accessibility of the technology outside of a laboratory or industrial setting. Elimination of this electrical equipment would allow electroporation to address small scale, far-reaching practical problems, such as destroying pathogens in contaminated water in developing nations.

Presented herein is an electrochemical cell configuration for performing electroporation without a pulse generator and minimal to no external power input. This electrochemical cell configuration is composed of an electrolyte flowing by a series of two adjacent, dissimilar metal electrodes separated by small insulators. When this configuration is in a non-equilibrium state, radially-varying electric fields emanating from the small insulators are present in the flowing electrolyte. These electric fields will electroporate biological cells suspended in the electrolyte or growing on the surface.

The crux of the concept presented is to utilize the ohmic potential drop in the electrolyte to perform electroporation. This ohmic potential drop establishes an electric field in the electrolyte, which at a given location is defined as the negative gradient of the local electric potential:

E=−∇φ

Therefore, to maximize the electric field in the electrolyte (1) the electric potential drop in the electrolyte has to be increased or (2) the electric potential drop needs to take place over a small distance. In electrolytic cells it is relatively easy to increase the potential drop in the electrolyte by adjusting the energy being input into the system. However, since the ultimate goal of this concept is to perform electroporation with no power input, a galvanic cell needs to be utilized, leaving little control of the potential drop in the electrolyte. Therefore, to increase the electric field magnitude in the electrolyte, the electrochemical cell geometry needs to be altered.

### Example 4

This example demonstrates the feasibility of a singularity-induced micro-electroporation; an electroporation configuration aimed at minimizing the potential differences required to induce electroporation by separating adjacent electrodes with a nanometer-scale insulator. In particular, this example presents a study aimed to understand the effect of (1) insulator thickness and (2) electrode kinetics on electric field distributions in the singularity-induced micro-electroporation configuration. A non-dimensional primary current distribution model of the micro-electroporation can still be performed with insulators thick enough to be made with micro-fabrication techniques. Furthermore, a secondary current distribution model of the singularity-induced micro-electroporation configuration with inner platinum electrodes and water electrolyte indicates that electrode kinetics do not inhibit charge transfer to the extent that probatively large potential differences are required to perform electroporation. These results indicate that singularity-induced micro-electroporation could be used to develop an electroporation system that consumes minimal power, making it suitable for remote applications such as the sterilization of water and other liquids.

The configuration, termed singularity-induced micro-electroporation, is composed of an electrolyte atop two adjacent electrodes separated by a small insulator. Application of a small potential difference between the adjacent electrodes results in a radially varying electric field emanating from the small insulator (FIG. 2A). Since it has been shown that applying an electric field along small portions of the cell membrane can induce electroporation, this radially varying electric field can be used to electroporate cells suspended in the electrolyte.

In order to implement the micro-electroporation channel, or other devices utilizing singularity-induced micro-electroporation, the practical feasibility of the configuration needs to be further analyzed. Understanding the effect of (1) insulator thickness and (2) electrode kinetics on electric field distributions in the singularity-induced micro-electroporation configuration is particularly important.

The insulator is the smallest feature in the singularity-induced micro-electroporation configuration. Because of this, it is one of the factors limiting the implementation of devices that utilize the singularity-induced micro-electroporation configuration. The effect of insulator thickness on electric field distribution in the singularity-induced micro-electroporation configuration needs to be analyzed to ensure that insulators thick enough to be created with micro-fabrication techniques can generate electroporation inducing electric field magnitudes at small potential differences.

In order to perform singularity-induced micro-electroporation with only a minimal power source (such as a battery), a direct current must be transferred from the electrodes to the electrolyte via electrochemical reactions. Because of this, the kinetics of the electrochemical reactions at the electrodes can inhibit current transfer. For singularity-induced micro-electroporation, the primary implication of inhibited current transfer is that prohibitively large potential differences could be required to generate electroporation inducing electric fields magnitudes. In order to ensure that this is not the case, the effect of electrode kinetics on electric field magnitudes in the singularity-induced micro-electroporation configuration need to be examined.

In this example we present (1) a modified, non-dimensional, primary current distribution model to analyze the effect of insulator thickness on the micro-electroporation channel, and (2) a secondary current distribution model of the singularity-induced micro-electroporation configuration with platinum electrodes and water electrolyte. The primary purpose of these models is to further assess the feasibility of singularity-induced micro-electroporation. Additionally, the secondary current distribution model is used to investigate the effect of water conductivity and applied voltage on the electric field distribution, and power input of the singularity-induced micro-electroporation configuration.

### Modified, Non-Dimensional, Primary Current Distribution Model for Analyzing the Effect of Insulator Thickness on the Micro-Electroporation Channel

Our previously developed, two-dimensional, steady-state, primary current distribution model was non-dimensionalized to analyze the effect of insulator thickness on the electric field in the electrolyte of the micro-electroporation channel.

Since this model neglects surface and concentration losses at the electrode surfaces, it is governed by the Laplace equation:

∇²φ=0

where φ is the electric potential. Furthermore, electrode surfaces are assumed to be at a constant potential, making the boundary conditions at the adjacent electrode surfaces:

φₐ=φ_{diff}

φ_{c}=0

where φₐ and φ_{c} are the potentials at the anode and cathode, respectively, φ_{diff} is the potential difference between the them. The remaining boundaries are insulation/symmetry boundaries and are governed by:

∇φ=0

Substituting the non-dimensional variables:

Φ=φ/φ_{diff}; X=x/L; Y=y/H

into the Laplace equation in two-dimensional Cartesian coordinates yields:

$$\frac{{\partial }^{2} Φ}{\partial {X}^{2}} + {( \frac{L}{H} )}^{2} \frac{{\partial }^{2} Φ}{\partial {Y}^{2}} = 0$$

In the above relations, L is the active electrode length and H is half of the height of the micro-electroporation channel. Defining the non-dimensional geometry parameter (aspect ratio):

$$A = \frac{H}{L}$$

the non-dimensional Laplace equation becomes:

$$\frac{{\partial }^{2} Φ}{\partial {X}^{2}} + \frac{1}{{A}^{2}} \frac{{\partial }^{2} Φ}{\partial {Y}^{2}} = 0$$

Substitution of the non-dimensional variables into the boundary conditions yields:

Φₐ=1; Φ_{c}=0; ∇Φ=0

Finally, the non-dimensional insulator thickness (relative insulator thickness) is defined as:

$$I = \frac{i}{L}$$

### Model Solution.

The non-dimensional primary current distribution model is characterized by the aspect ratio (A) and relative insulator thickness (I). A parametric study was performed by varying I and A in a series of models. In each model, the non-dimensional potential distribution was solved for using a finite difference method implemented in MATLAB (R2007a version 7.4). A non-dimensional electric field defined as:

NDE=∇Φ

was calculated using the non-dimensional potential distribution.

### Secondary Current Distribution Model of Singularity-Induced Micro-Electroporation

A two-dimensional, steady-state, secondary current distribution model was developed to analyze the effects of electrode kinetics on singularity-induced micro-electroporation Like primary current distribution models, secondary current distribution models account for electric field effects from ohmic losses in the bulk electrolyte, and are therefore governed by the Laplace equation (Eqn. 1) in that region. However, unlike primary current distribution models, secondary current distribution models account for kinetic losses at the electrode surfaces. Since kinetic losses strongly depend on the potential at an electrode surface, the boundary conditions at the adjacent electrode surfaces are:

jₐ=−σ∇φₐ=f(η_{s,a})

j_{c}=−σ∇φ_{c}=f(η_{s,c})

where ja and jc are the current densities at the anode and cathode, respectively, σ is the conductivity of the bulk electrolyte, and η_{s,a} and η_{s,c} are the surface overpotentials at the anode and cathode, respectively. Overpotential represents a departure from the equilibrium potential at an electrode surface, and is defined as:

η=φ−E⁰

where E⁰ is the equilibrium potential for an electrochemical reaction at standard state, typically 293 K at 1 atm.

### Electrode Kinetics Model.

Neglecting concentration losses, the relationship between current and potential at electrode surfaces is commonly described by a modified version of the Butler-Volmer model:

$$j = {j}_{0} [ exp \frac{ {α}_{a} F {η}_{s}}{RT} - exp \frac{- {α}_{c} F {η}_{s}}{RT} ]$$

Conceptually, the first term describes the anodic (reduction) contribution to the net current at a given potential, while the second term describes the cathodic (oxidation) contribution to the net current. With that in mind, the variables in the Butler-Volmer model are:

j₀, the exchange current density. The exchange current density is the current density where the anodic and cathodic contributions are equal, resulting in no net current.
αₐ and α_{c}, the anodic and cathodic transfer coefficients, which respectively describe the energy required for each reaction to occur.
ηₛ, the surface overpotential, the deviation of the electrode potential from its equilibrium potential.
F, the Faraday constant (96500 C/mol).
R, the universal gas constant (8.314 J/mol-K).
T, the temperature of the electrode reaction (K).

The exchange current density, and the anodic and cathodic transfer coefficients are determined experimentally, typically by fitting current-potential data to the Butler-Volmer model. However, in some cases, it is more convenient to fit current potential data to simpler forms (i.e., linear).

### Development of the Current Density Boundary Conditions.

A voltage must be applied to the cell suspension to generate an electric field for electroporation. Because of potential losses due to irreversibilities (Eₗₒₛₛ), the applied voltage (Vₐₚₚₗ) must be greater than the equilibrium potential (E_{eq}) of the electrochemical cell [33]:

Vₐₚₚₗ=E_{eq}+Eₗₒₛₛ

The equilibrium potential of the electrochemical cell is the difference between the anode and cathode reduction equilibrium potentials at standard state (E⁰ₐ and E⁰_{c}, respectively):

E_{eq}=Eₐ⁰−E_{c}⁰

Irreversible losses have three classifications: 1) surface losses from sluggish electrode kinetics; 2) concentration losses due to mass-transfer limitations; and 3) ohmic losses in the electrolyte.

Since concentration losses are neglected in secondary current distribution models, the irreversible losses can be represented as:

Eₗₒₛₛ=η_{s,a}−η_{s,c}+Δφₒₕₘ

where Δφₒₕₘ is the ohmic loss in the electrolyte, and can be further decomposed to:

Δφₒₕₘ=φₐ−φ_{c}

Combining equations results in:

Vₐₚₚₗ=E_{eq}+η_{s,a}−η_{s,c}+φₐ−φ_{c}

which provides a more detailed relation for the voltage that must be applied to the electrochemical cell to compensate for irreversible losses. Since kinetic models provide the net current density at an electrode surface as a function of surface overpotential, the equation above can be separated to obtain the surface overpotentials at the anode and cathode:

η_{s,a}=Vₐₚₚₗ−E_{eq}−φₐ

η_{s,c}=−φ_{c}

Substituting these relations into the modified version of the Butler-Volmer equation relates the surface potentials at the anode and cathode to their respective current densities, enabling an implicit numerical solution.

$${j}_{a} = {j}_{0 , a} [ exp \frac{{α}_{a , a} F {η}_{s , a}}{RT} - exp \frac{- {α}_{c , a} F {η}_{s , a}}{RT} ]$$

$${j}_{c} = {j}_{0 , c} [ exp \frac{{α}_{a , c} F {η}_{s , c}}{RT} - exp \frac{- {α}_{c , c} F {η}_{s , c}}{RT} ]$$

### Model Parameters.

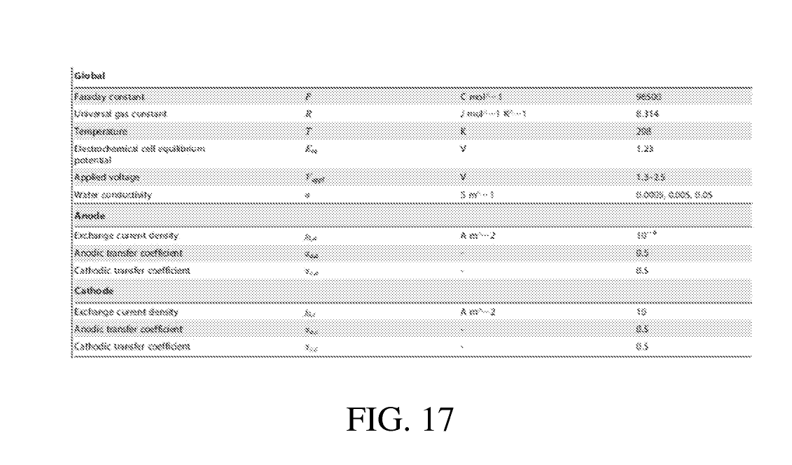

The parameters used in the secondary current distribution model are outlined in the table in FIG. 17.

The secondary current distribution model domain is shown in FIG. 4(b). The domain is 10 microns long, has a 100 nanometer thick insulator, and is 20 microns high. Since previous results show that decreasing domain height exponentially increases electric field magnitudes, the height of the domain was made sufficiently large to determine the minimum electric field magnitudes that can be generated when accounting for electrode kinetics.

Since we would like to use the singularity-induced micro-electroporation configuration for water sterilization, the bulk electrolyte is water. The electrical conductivity of water typically varies between 0.0005 and 0.05 S/m.

The anode and cathode are modeled as inert platinum electrodes. In water, the electrochemical reactions that take place at the electrode surfaces are identical to those in water electrolysis. At the anode, water is oxidized:

2H₂O [symbol] O₂(gas)+4H⁺(aq)+4e⁻

Under standard conditions, this reaction has a reduction equilibrium potential (E⁰ₐ) of 1.23 V and an exchange current density (j_{a,0}) of 1028 A/m². Additionally, the transfer coefficients (α_{a,a} and α_{a,c}) were assumed to be 0.5. At the cathode, water is reduced:

4H₂O+4e⁻ [symbol] 2H₂(gas)+4OH⁻(aq)

Under standard conditions, this reaction has a reduction potential (E⁰_{c}) of −0.83 V and an exchange current density (j_{c,0}) of 10 A/m2. Similar to the water oxidation reaction at the anode, the transfer coefficients (α_{c,a} and α_{c,c}) were assumed to be 0.5. Therefore, the net reaction in the platinum-water singularity-induced micro-electroporation system is:

2H₂O [symbol] 2H₂(gas)+O₂(gas)

Under standard conditions, this reaction has an equilibrium potential (E_{eq}) of 2.06 V that must be exceeded to generate an electric field distribution in the water.

It should be noted that since saline is a water based solution, these electrochemical reactions are also applicable to a more traditional electroporation system. Therefore, this secondary current distribution model could easily be modified to analyze singularity-induced micro-electroporation in a saline solution by changing the bulk electrolyte conductivity.

### Model Solution.

The secondary current distribution model is affected by the conductivity of the water electrolyte(s) and voltage applied (Vₐₚₚₗ) to the electrochemical cell. A parametric study was performed by varying these parameters in a series of models. In each model, the potential distribution was solved for using the finite element analysis software COMSOL Multiphysics 4.0a. The electric field defined as:

E=∇φ

was calculated using the potential distribution. Furthermore, by integrating the current density at the anode or cathode boundary, the total current (jt ot) through the model was determined. Using the total current through the model, the power input defined as:

P=jₜₒₜVₐₚₚₗ

was calculated.

### Non-Dimensional Primary Current Distribution Model for Analyzing the Effect of Insulator Thickness

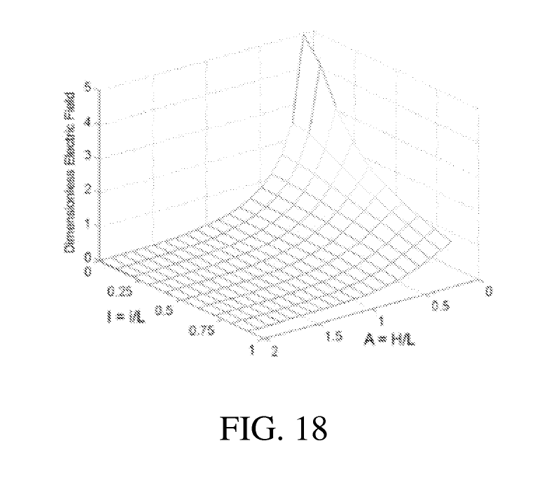

The results of the non-dimensional primary current distribution model show that decreasing the relative insulator thickness (I) increases the magnitude of the non-dimensional electric field (NDE) at the center of the micro-electroporation channel (FIG. 18). More specifically, the extent of the increase in the nondimensional electric field magnitude due to relative insulator thickness depends on the aspect ratio (A). At low aspect ratios, decreasing relative insulator thickness substantially increases the non-dimensional electric field. Decreasing the relative insulator thickness from 0.9 to 0 (singularity) at an aspect ratio of 0.1 results in a 413% increase in non-dimensional electric field magnitude. Conversely, at high aspect ratios, decreasing the relative insulator thickness negligibly increases the non-dimensional electric field. At an aspect ratio of 2, decreasing the relative insulator thickness from 0.9 to 0 results in a 115% increase in non-dimensional electric field magnitude.

### Secondary Current Distribution Model of Singularity-Induced Micro-Electroporation—Effect of Water Conductivity and Applied Voltage on Electric Field Distribution.

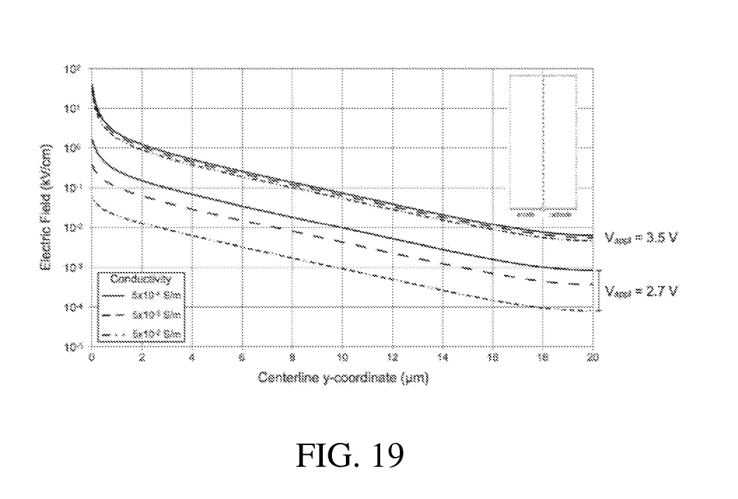

The conductivity of the water (s) and the applied voltage (Vappl) both influence the electric field distribution in the singularity-induced micro-electroporation configuration. At applied voltages lower than 3.2 V, low conductivity water contains substantially larger electric field magnitudes than high conductivity water (FIG. 19). For example, at an applied voltage of 2.7 V, the electric field magnitudes at the center of the insulator are 0.06, 0.38, and 1.64 kV/cm at water conductivities of 0.05, 0.005, and 0.0005 S/m, respectively. Furthermore, at applied voltages lower than 2.8 V, increasing the applied voltage exponentially increases electric field magnitudes in the water. Conversely, at applied voltages higher than 2.8 V, the electric field distribution becomes constant and independent of water conductivity. At an applied voltage of 3.5 V, the electric field magnitudes at the center of the insulator are 26.4, 33.1, and 39.8 kV/cm at water conductivities of 0.05, 0.005, and 0.0005 S/m, respectively.

### Effect of Water Conductivity and Applied Voltage on Power Input.

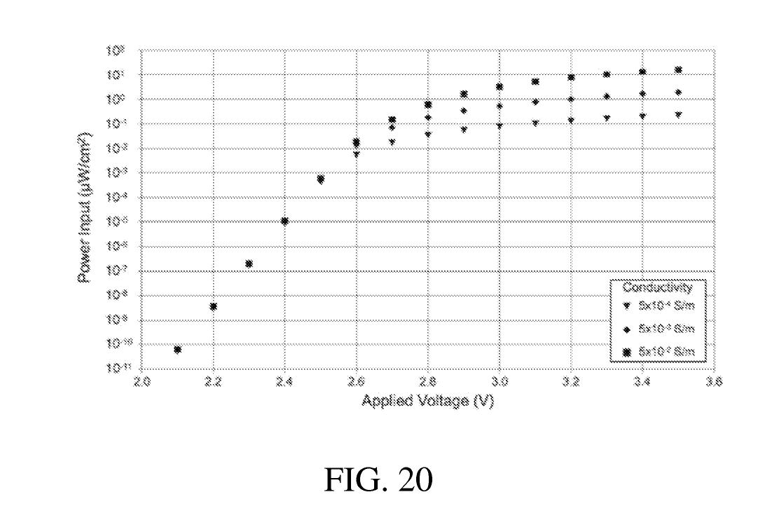

The power input to the singularity-induced micro-electroporation configuration is also dependent on the conductivity of the water and the applied voltage (FIG. 20). At applied voltages less than ˜2.6 V, power input is independent of water conductivity and increases exponentially with applied voltage. For example, at an applied voltage of 2.4 V, the powers input to the singularity-induced micro-electroporation configuration are 1.09, 1.05, and 0.92×10⁻⁵ μW/cm² at water conductivities of 0.05, 0.005, and 0.0005 S/m, respectively. Conversely, at applied voltages greater than ˜2.6 V, the power input becomes constant and is highly dependent on the water conductivity. A singularity-induced micro-electroporation configuration with low conductivity water (0.0005 S/m) requires the least power input, 0.23 μW/cm² at an applied voltage of 3.5 V. The power input required by the singularity-induced micro-electroporation configuration substantially increases with water conductivity. Configurations with 0.005 and 0.05 S/m water conductivities require 1.93 and 16.20 μW/cm², respectively.

### Effect of Insulator Thickness

The results of the non-dimensional primary current distribution model demonstrate the practical feasibility of the micro-electroporation channel. In our previous work, we predicted that increasing the insulator thickness would decrease the electric field magnitudes throughout the electrolyte of the micro-electroporation channel. While our results quantitatively support this prediction, they also indicate that electroporation inducing electric fields can be generated with insulators thick enough to be created with micro-fabrication techniques. For example, applying a 0.5V potential difference in a micro-electroporation channel with an active electrode length (L) of 10 mm, micro-electroporation channel height (2H) of 2 mm, and insulator thickness (i) of 100 nm (non-dimensional data for A=0.1, I=0.01), can generate electric field magnitudes in excess of 10 kV/cm, which are sufficient for inducing irreversible electroporation. Numerous lithographic techniques are capable of producing sub-100 nm features, and could be used to create the insulators in a micro-electroporation channel. Immersion lithography is a photolithography enhancement technique that places a liquid with a refractive index greater than one between the final lens and wafer. Current immersion lithography tools are capable of creating feature sizes below 45 nm. Additionally, electron beam lithography, a form of lithography that uses a traveling beam of electrons, can create features smaller than 10 nm.

### Secondary Current Distribution Model of Singularity-Induced Micro-Electroporation

Electrochemical reactions must transfer a direct current from the electrodes to the electrolyte to perform singularity-induced micro-electroporation. The kinetics of electrochemical reactions can inhibit current transfer and potentially necessitate prohibitively large potential differences to generate electroporation-inducing electric field magnitudes. Therefore, to adequately analyze the feasibility of implementing a singularity-induced micro-electroporation system, the effect of electrode kinetics on electric field magnitudes must be understood. The secondary current distribution model of the singularity-induced micro-electroporation configuration with platinum electrodes and water electrolyte accounts for electrode kinetics. The results of this model: (1) demonstrate the practical feasibility of implementing a singularity-induced micro-electroporation system, (2) predicts the upper limit to the electric field magnitudes of the system, and (3) provides data for optimizing the power input necessary to obtain a desired electric field distribution.

The practical feasibility of creating a singularity-induced micro-electroporation system is demonstrated by the results of the secondary current distribution model with platinum electrodes and water electrolyte. The results show that electric fields in excess of those required to induce reversible (1-3 kV/cm) and irreversible (10 kV/cm) electroporation can be generated in water with platinum electrodes. For instance, in water with a conductivity of 0.0005 S/m, an applied voltage as low as 2.8 V (0.7 V larger than E_{eq}) can generate electric fields sufficient to induce reversible electroporation near the insulator surface. Increasing the applied voltage by 0.1 V generates electric fields capable of inducing irreversible electroporation near the insulator surface, and reversible electroporation at distances up to ˜0.7 μm from the insulator. Although lower electric field magnitudes are present in higher conductivity water (0.005 or 0.05 S/m), minor increases in applied voltage result in similar reversible and irreversible electroporation inducing electric fields.

The trend shown in FIG. 19 indicates that there is an upper limit to the electric field magnitudes that can be generated in the singularity-induced micro-electroporation system. For this system, the low exchange current density of the anode electrochemical reaction (j_{0,a}) limits the current through the system. As a result, as the applied voltage increases, the water conductivity has less of an influence on the electric field distribution. Furthermore, at large applied voltages, increasing the applied voltage negligibly changes the electric field distribution, indicating the upper limit of the electric field magnitudes that can be generated with this system. Close to the insulator, the electric field magnitudes at the upper limit are well above the magnitudes required to induce reversible and irreversible electroporation. However, if large electric field magnitudes are required away from the insulator, the upper limit may become an important design consideration.

The secondary current distribution model of singularity-induced micro-electroporation can be used to optimize the power input to the system. As previously noted, at large applied voltages, water conductivity is negligibly influential and the electric field distribution becomes constant with increasing applied voltage (FIG. 19). FIG. 20 shows that while power input also becomes constant at large applied voltages, it is substantially affected by water conductivity. In general, low conductivity water (0.0005 S/m) generates the largest electric field magnitudes with the least power input, and high conductivity water (0.05 S/m) generates the smallest electric field magnitudes with the most power input. Therefore, decreasing the water conductivity is the most effective method for optimizing the power input to the system.

It should be noted that the methodology used for developing the secondary current distribution model of singularity-induced micro-electroporation could be used to model a variety of electroporation devices. With appropriate electrode kinetics parameters, numerous electrode materials and electroporation configurations could be examined. These models would aid in experimental studies by providing electric field distributions throughout the electrolyte. Additionally, they would facilitate the optimal design of electroporation systems for a variety of applications.

The singularity-induced micro-electroporation configuration offers numerous advantages over traditional macro and micro-electroporation devices. In electroporation devices with facing electrodes, a cell's proximity has no bearing on the electric field magnitude it will experience. Conversely, in a singularity-induced micro-electroporation configuration, the electric field magnitude experienced by a cell is dictated by the gap between the cell and the surface of the configuration. Because of this, cell size does not affect the potential difference required to achieve a desired electric field.

Another advantage of the singularity-induced micro-electroporation configuration over traditional macro and micro-electroporation devices is that less electrical equipment is required. Traditional macro and micro-electroporation devices require a pulse generator and power supply. However, by placing singularity-induced micro-electroporation configurations in series, as is done in the micro-electroporation channel, the need for a pulse generator is eliminated. Furthermore, as validated by the secondary current distribution model, only a small potential difference is required. Because of this, only a minimal power source (such as a battery) is needed.

The practical feasibility of singularity-induced micro-electroporation systems were assessed by examining the effect of insulator thickness and electrode kinetics on generated electric field distributions. Two models were developed to understand these effects: (1) a modified, non-dimensional, primary current distribution model of a micro-electroporation channel, and (2) a secondary current distribution model of the singularity-induced micro-electroporation configuration with platinum electrodes and water electrolyte.

A previously developed, non-dimensional, primary current distribution model was modified to analyze the effect of insulator thickness on the electric field distribution of a micro-electroporation channel. Increasing the insulator thickness exponentially reduces the electric field magnitude directly above the center of the insulator and inhibits the permeation of high-strength electric fields in the electrolyte. However, high-strength electric fields can still be generated with insulators thick enough to be created with MEMS manufacturing techniques. Therefore, insulator thickness does not inhibit the practical feasibility of creating singularity-induced micro-electroporation systems.

A secondary current distribution model of the singularity-induced micro-electroporation configuration with platinum electrodes and water electrolyte was developed to examine the effect of electrode kinetics on the electric field distribution in the water. The results of this model show that electric field magnitudes in excess of those required to induce reversible (1-3 kV/cm) and irreversible (10 kV/cm) electroporation can be generated in water with platinum electrodes. This further substantiates the practical feasibility of implementing a singularity-induced micro-electroporation device. Additionally, the secondary current distribution model shows that at low applied voltages, significantly larger electric field magnitudes are present in lower conductivity water. Initially, as the applied voltage increases there is an exponential increase in electric field magnitudes in the water. However, at large applied voltages, increasing the applied voltage negligibly changes the electric field magnitudes, regardless of water conductivity. Furthermore, at large applied voltages, the required power input is highly dependent on the conductivity of the water. Therefore, it can be concluded that low conductivity water generates the largest electric field magnitudes with the least power input, and high conductivity water generates the smallest electric field magnitudes with the most power input.

### Example 5

This example demonstrates the feasibility of creating a self-powered (galvanic) electroporation device using the singularity-induced nano-electroporation configuration. Using this configuration, the electric field in a galvanic electrochemical cell can be amplified and used for electroporation. A secondary current distribution model of a self-powered electroporation device shows that the device can create both reversible and irreversible electroporation-inducing electric field magnitudes, and generate a small amount of power. The generated power could be also harvested for a variety of applications.

Because the singularity-induced nano-electroporation configuration can generate high-strength electric fields with small potential differences, we believe it is possible to use the configuration to create an electroporation device that does not require an external power source. Presented is a galvanic electroporation device, termed the self-powered nano-electroporation device. The self-powered nano-electroporation device will use the singularity-induced nano-electroporation configuration to amplify the electric field distribution created by the ohmic drop of a galvanic electrochemical cell. This electric field distribution can be used to perform electroporation.

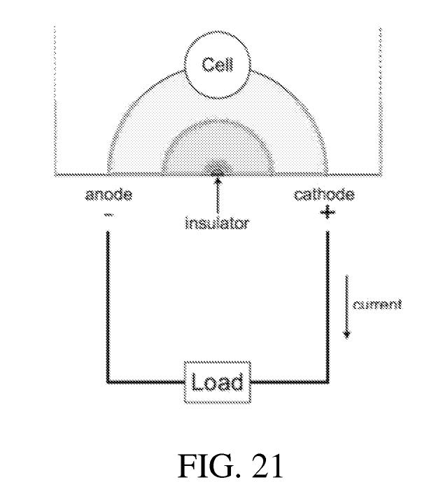

Electroporation devices are electrochemical cells that aim to maximize the ohmic drop in the electrolyte to generate larger electric field magnitudes. To date, all electroporation devices have been electrolytic electrochemical cells—electric current is supplied to generate a significant ohmic drop and resulting electric field in the electrolyte. Conversely, galvanic electrochemical cells convert chemical reactions to electric current. These chemical reactions typically occur at two dissimilar material electrodes, an anode and cathode, where oxidation and reduction occur, respectively. The anode and cathode are separated by an electrolyte that conducts ionic current between them. When electric current is drawn from a galvanic electrochemical cell, a small potential distribution develops in the electrolyte, resulting in an electric field that can be used to perform electroporation (FIG. 21).

Here we present a secondary current distribution model of a self-powered nano-electroporation device composed of an aluminum anode, air cathode, and water electrolyte. The primary purpose of this model is to demonstrate the feasibility of self-powered nano-electroporation by showing the generation of electroporation-inducing electric field magnitudes. In particular, the model is used to determine the effect of water conductivity and load voltage on the electric field distribution in the self-powered nano-electroporation device. Furthermore, because the self-powered nano-electroporation device is a galvanic electrochemical cell, and power output of the device is also investigated.

A secondary current distribution model was developed to determine the electric field magnitudes and power output characteristics of a self-powered nano-electroporation device utilizing aluminum-air chemistry.

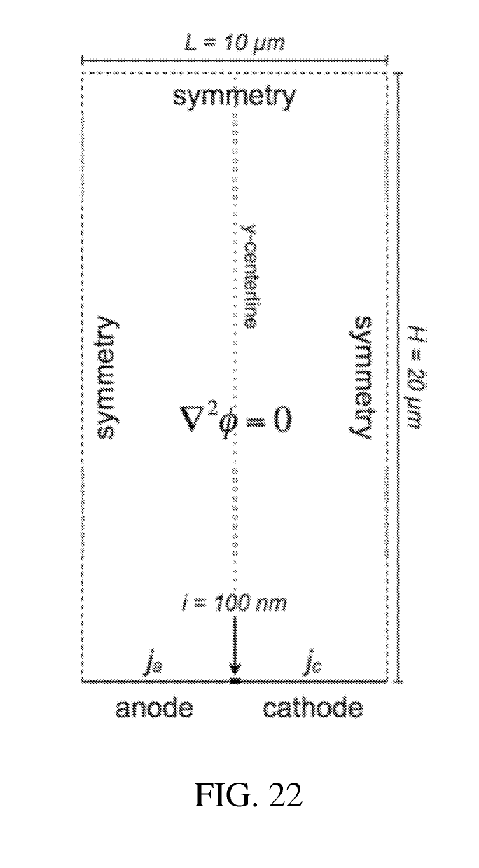

The secondary current distribution model domain is shown in FIG. 22. Previous results have shown that decreasing the aspect ratio of the model domain significantly increases the electric field magnitudes throughout the domain. Therefore, to minimize geometric electric field enhancement, a model domain with an aspect ratio of 2, corresponding to a domain height and length of 20 and 10 μm, respectively, was used for the secondary current distribution model. Additionally, a 100 nm thick insulator, large enough to be created with micro-fabrication techniques, was used.

Secondary current distribution models account for ohmic drops in the bulk electrolyte and kinetic losses at electrode surfaces. Therefore, the bulk electrolyte region is governed by the Laplace equation:

∇²φ=0

where φ is the electric potential. To account for kinetic losses, which depend on the potential at the electrode surface, the boundary conditions at the adjacent electrodes are:

jₐ=−σ∇φₐ=f(η_{s,a})

j_{c}=−σ∇φ_{c}=f(η_{s,c})

where ja and jc are the current densities at the anode and cathode, respectively, σ is the conductivity of the bulk electrolyte, and ηs,a and ηs,c are the surface overpotentials at the anode and cathode, respectively. Overpotential represents a deviation from the equilibrium potential at an electrode surface, and is defined as:

η=φ−E⁰

where E⁰ is the equilibrium potential for an electrochemical reaction at standard state, typically 293 K at 1 atm. The remaining boundaries are insulation/symmetry boundaries and are governed by:

∇φ=0

The relationship between current density and potential at the electrode surfaces is typically obtained by fitting experimental data. In water, the primary electrochemical reaction at the aluminum anode is:

Al+3OH⁻ [symbol] Al(OH)₃+3e⁻

Furthermore, in water, there is an additional, parasitic, reaction at the aluminum anode:

$$Al + 3 {H}_{2} O ⇔ {Al ( OH )}_{3} + \frac{3}{2} {H}_{2}$$

Accounting for these reactions, the kinetic parameters of the aluminum anode were determined by fitting a polarization curve to the Butler-Volmer equation:

$${j}_{a} = {j}_{0 , a} [ exp \frac{{α}_{a , a} F {η}_{s , a}}{RT} - exp \frac{- {α}_{a , c} F {η}_{s , a}}{RT} ]$$

Conceptually, the first term describes the anodic (reduction) contribution to the net current at a given potential, while the second term describes the cathodic (oxidation) contribution to the net current. With that in mind, the variables in the Butler-Volmer model are: j_{0,a}, the anode exchange current density. The exchange current density is the current density where the anodic and cathodic contributions are equal, resulting in no net current. α_{a,a} and α_{a,c}, the anodic and cathodic transfer coefficients at the anode, which respectively describe the energy required for each reaction to occur.

### Nomenclature

ηs,a the surface overpotential at the anode, the deviation of the electrode potential from its equilibrium potential.
F, the Faraday constant (96500 C/mol).
R, the universal gas constant (8.314 J/mol-K).
T, the temperature of the electrode reaction (K).

The electrochemical reaction at the air cathode in water is:

O₂+2H₂O+4e⁻ [symbol] 4OH

The current-potential relation for this reaction was determined by linearly fitting a polarization curve for a Yardney AC51 air cathode:

j_{c}=aη_{s,c}+b

For a galvanic electrochemical cell, the voltage delivered is going to be less than the equilibrium potential of the electrochemical cell due to irreversible losses:

V_{del}=E_{eq}−Eₗₒₛₛ

The equilibrium potential of the electrochemical cell is the difference between the cathode and anode reduction equilibrium potentials at standard state (Ea⁰ and Ec⁰, respectively):

E_{eq}=E_{c}⁰−Eₐ⁰

Irreversible losses have three classifications: 1) surface losses from sluggish electrode kinetics; 2) concentration losses due to mass-transfer limitations; and 3) ohmic losses in the electrolyte.

Since concentration losses are neglected in secondary current distribution models, the irreversible losses can be represented as:

Eₗₒₛₛ=η_{s,a}−η_{s,c}+Δφₒₕₘ

where Δφₒₕₘ is the ohmic loss in the electrolyte, and can be further decomposed to:

Δφₒₕₘ=φₐ−φ_{c}

Combining equations:

V_{del}=E_{eq}−η_{s,a}+η_{s,c}−φₐ+φ_{c}

which provides a more detailed relation for the voltage that must be applied to the electrochemical cell to compensate for irreversible losses. Since kinetic models provide the net current density at an electrode surface as a function of surface overpotential, the equation above can be separated to obtain the surface overpotentials at the anode and cathode:

η_{s,a}=E_{eq}−V_{del}−φₐ

η_{s,c}=−φ_{c}

Substituting equations into the current-potential relations for the anode and cathode, respectively, enables an implicit numerical solution.

The results of the secondary current distribution model are affected by the conductivity of the bulk electrolyte (α) and the load voltage (V_{load}), which regulates the amount of current drawn from the device (decreasing the load voltage increase the current drawn). A parametric study was performed by varying the conductivity and load voltage in a series of models. Table 2 contains the parameters used in the secondary current distribution models.

TABLE 2
Secondary current distribution model parameters.
Global

| | | |
|---|---|---|
| F | C/mol | 96500 |
| R | J/mol-K | 8.314 |
| T | K | 298 |
| E_{eq} | V | 1.41 |
| Vₐₚₚₗ | V | 0.5-1.4 |
| σ | S/m | 5e−4, 5e−3, 5e−2 |

Anode

| | | |
|---|---|---|
| j_{0,a} | A/m² | 816.87 |
| α_{a,a} | — | 0.08767 |
| α_{c,a} | — | 0.2134 |

Cathode

| | | |
|---|---|---|
| a | A/m²-V | 2270 |
| b | A/m² | −24.2 |

In each model, the potential distribution was solved for using the finite element analysis software COMSOL Multiphysics 4.0a. The electric field, defined as:

E=∇φ

was calculated using the potential distribution. Furthermore, by integrating the current density at the anode or cathode boundary, the total current through the model domain was determined. Using the total current through the domain, the power output, defined as:

P=jₜₒₜV_{del}

was calculated.

The goal of every electroporation device is to generate electric field magnitudes that are capable of inducing electroporation, which requires substantial ohmic drops in the electrolyte. In regards to the self-powered nano-electroporation configuration, (1) decreasing the conductivity (α) and (2) decreasing the load voltage (Vload) (increasing the current drawn from the configuration) increases the ohmic drop in the electrolyte.

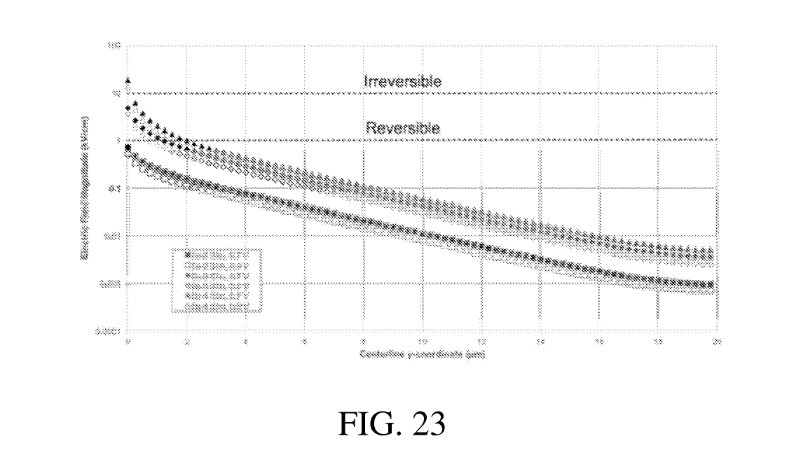

The secondary current distribution model shows that decreasing the electrolyte conductivity increases the electric field magnitudes in the self-powered nano-electroporation configuration (FIG. 23). Electroporation-inducing electric field magnitudes cannot be generated in water with a conductivity of 5e-2 S/m. However, water with a conductivity of 5e-3 S/m is capable of generating reversible electroporation-inducing electric field magnitudes (>1 kV/cm21) at load voltages of less than 1.2 V. The largest electric field magnitudes are present in water with a conductivity of 5e-4 S/m. At this conductivity, a load voltage as high as 1.3 V results in a maximum electric field magnitude of 2.68 kV/cm. Furthermore, at a load voltage of 0.9 V the maximum electric field in a configuration with a conductivity of 5e-4 S/m is 13.12 kV/cm, which is larger than the electric field magnitude required to induce irreversible electroporation (>10 kV/cm21).

For a given conductivity, the secondary current distribution model shows that decreasing the load voltage (increasing the current density drawn from the self-powered nano-electroporation configuration) increases the electric field magnitudes in the electrolyte (FIG. 23). Maximum electric field magnitudes of 3.48 and 4.82 kV/cm can be generated in water with a conductivity of 5e-3 S/m at load voltages of 0.9 and 0.7 V, respectively. However, at lower conductivities, the same load voltages generate substantially larger electric field magnitudes. Water with a conductivity of 5e-4 S/m is capable of generating maximum electric field magnitudes of 13.2 and 18.2 kV/cm at load voltages of 0.9 and 0.7 V, respectively. The reason for the discrepancy in electric field magnitudes between the water conductivities can be explained by examining the sources of potential losses in the self-powered nano-electroporation configuration. In a configuration with 5e-3 S/m conductivity water, the ohmic drop in the electrolyte is not the dominant potential loss in the configuration. The air cathode is non-polarizable relative to the anode. Therefore, to sustain the large currents required to generate an electric field in the electrolyte, a large overpotential must be present at the cathode surface. For this scenario, the overpotential at the cathode is the dominant potential drop in the configuration. This is not the case in a configuration with 5e-4 S/m conductivity water, where the dominant potential loss is the ohmic drop in the electrolyte, which results in larger electric field magnitudes.

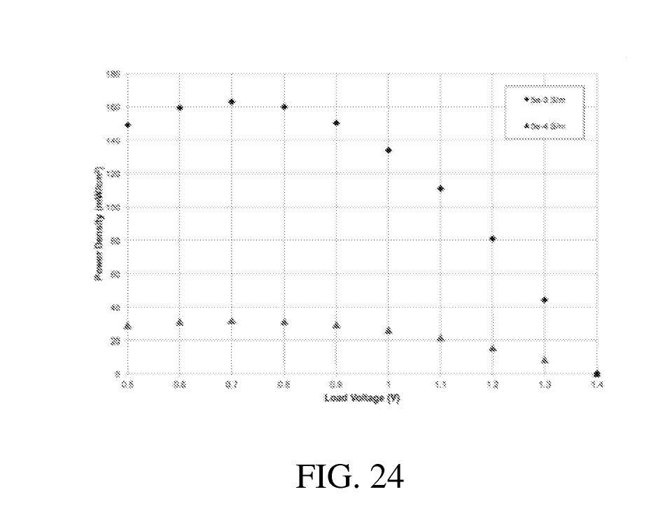

Since the self-powered electroporation configuration is a galvanic electrochemical cell, it can also generate a small amount of power (FIG. 24). FIG. 24 does not include power output data for 5e-2 S/m water because it could not generate electroporation-inducing electric field magnitudes. While power generation is not the primary purpose of the configuration, the power it generates could potentially be used in MEMS applications. As expected, configurations with 5e-3 S/m water produce the most power, while configurations with 5e-4 S/m water produce the least power. For both conductivities, the maximum power output occurs at a load voltage of 0.7 V. At this load voltage, power output densities of 163.07 and 31.85 mW/cm2 are produced in 5e-3 and 5e-4 S/m water, respectively. Therefore, as expected, configurations that result in the largest electric field magnitudes also produce the least power. Nonetheless, it may be possible to optimize the configuration to satisfy a set of given electric field and power output requirements.

It should be noted that the power output predicted for 5e-3 S/m conductivity water may be higher than would be experimentally observed. Polarization data for the air cathode only went up to 60 mA/cm2, and at a conductivity of 5e-3 S/m, the current generated by the device at low load voltages exceeded that value. Therefore, for those scenarios, the polarization data at the air cathode was extrapolated. The current densities for 5e-4 S/m water never exceeded 60 mA/cm2.

A secondary current distribution model of a self-powered nano-electroporation device composed of an aluminum anode, air cathode, and water electrolyte was developed to assess the theoretical feasibility of self-powered nano-electroporation. The model indicates that self-powered nano-electroporation is theoretically feasible. At sufficiently low electrolyte conductivities, the aluminum-air chemistry is capable of generating reversible and irreversible electroporation-inducing electric field magnitudes. Additionally, for a given electrolyte conductivity, decreasing the load voltage of (increasing the current drawn from) the self-powered nano-electroporation device increases the electric field magnitudes in the electrolyte. Finally, it is possible to generate a small amount of power from the self-powered electroporation device.

### CONCLUSION

The foregoing description of the invention has been presented for purposes of illustration and description. It is not intended to be exhaustive or to limit the invention to the precise form disclosed. Other modifications and variations may be possible in light of the above teachings. The embodiments were chosen and described in order to best explain the principles of the invention and its practical application, and to thereby enable others skilled in the art to best utilize the invention in various embodiments and various modifications as are suited to the particular use contemplated. It is intended that the appended claims be construed to include other alternative embodiments of the invention; including equivalent structures, components, methods, and means.

It is to be appreciated that the Detailed Description section, and not the Summary and Abstract sections, is intended to be used to interpret the claims. The Summary and Abstract sections may set forth one or more, but not all exemplary embodiments of the present invention as contemplated by the inventor(s), and thus, are not intended to limit the present invention and the appended claims in any way.

What is claimed is:

1. A singularity-based electrode configuration, comprising:
an anode electrode;
a cathode electrode; and
an insulator disposed between the anode electrode and the cathode electrode, wherein the anode electrode, insulator, and cathode electrode are positioned co-planar with respect to one another.

2. The singularity-based electrode configuration of claim 1, further comprising:
an ionic substance in contact with the anode electrode, insulator, and cathode electrode.

3. The singularity-based electrode configuration of claim 1, wherein the insulator separates the anode electrode from the cathode electrode by between five nanometers and five microns.

4. The singularity-based electrode configuration of claim 1, wherein the insulator separates the anode electrode from the cathode electrode by between 50 nanometers and two microns.

5. The singularity-based electrode configuration of claim 1, wherein the insulator separates the anode electrode from the cathode electrode by about 100 nm.

6. The singularity-based electrode configuration of claim 1, wherein the insulator separates the anode electrode from the cathode electrode by less than 100 nm.

7. The singularity-based electrode configuration of claim 1, further comprising:
a power supply selected from a group consisting of: a DC power supply, an AC power supply, a pulsed potential power supply, a current pulse power supply, and an electrolytic reaction involving the electrodes and an ionic substance;
wherein the power supply is connected to the electrodes.

8. The singularity-based electrode configuration of claim 1, further comprising:
a substance of interest selected from the group consisting of: an ionic solution containing cells, tissue in vitro, and tissue in vivo.

9. A micro-electroporation channel configuration, comprising:
an anode electrode;
a cathode electrode; and
an insulator disposed between the anode electrode and the cathode electrode, wherein the anode electrode, insulator, and cathode electrode are positioned co-planar along one side of the micro-electroporation channel.

10. The micro-electroporation channel configuration of claim 9, further comprising:
an electrolyte flowing through the channel over the anode electrode, insulator, and cathode electrode.

11. The micro-electroporation channel configuration of claim 9, wherein the insulator separates the anode electrode from the cathode electrode by between 50 nanometers and two microns.

12. The micro-electroporation channel configuration of claim 9, further comprising:
a power source selected from a group consisting of: a pulsed potential, an AC potential, and an electrolytic reaction involving the electrodes and an ionic solution.

13. The micro-electroporation channel configuration of claim 12, wherein the ionic solution is a physiological solution that contains cells, live tissue, or dead tissue.

14. The micro-electroporation channel configuration of claim 9, further comprising:
a second anode electrode positioned on the opposite side of the channel relative to the first anode electrode;
a second cathode electrode positioned on the opposite side of the channel relative to the first cathode electrode; and
a second insulator disposed between the second anode electrode and the second cathode electrode, wherein the second anode electrode and the second cathode electrode are co-planar with respect to one another.

15. A method of micro-electroporation, the method comprising:
providing a micro-electroporation channel including a series of co-planar anode electrodes and cathode electrodes, wherein adjacent anode electrodes and cathode electrodes are separated by an insulator;
flowing an electrolyte through the micro-electroporation channel;
flowing a cell through the micro-electroporation channel; and
applying a potential difference between adjacent anode electrodes and cathode electrodes.

16. The method of claim 15, further comprising:
alternating the flow rate of the electrolyte through the micro-electroporation channel.

17. The method of claim 15, wherein each insulator separates the anode electrode from the adjacent cathode electrode by between 50 nanometers and two microns.

18. The method of claim 15, further comprising:
coupling the anode electrodes and the cathode electrodes to a power source selected from the group consisting of: a DC power supply, an AC power supply, a pulsed potential power supply, a current pulse power supply, and an electrolytic reaction involving the electrodes and an ionic substance.

19. A method of water sterilization comprising the method of claim 15.

20. A method of cell transfection comprising the method of claim 15.
